(19) United States
(12) Patent Application Publication
(10) Pub. No.: US 2009/0326027 A1
(43) Pub. Date: Dec. 31, 2009

(54) N-Phenyl-2-0X0-1,4-Diazaspiro [4.5] Dec-3-EN-1-YL Acetamide Derivatives And Their Use As Glycine Transporter Inhibitors

(76) Inventors: Steven Coulton, Essex (GB); Howard Robert Marshall, Essex (GB); David John Nash, Essex (GB); Roderick Alan Porter, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/282,537

(22) PCT Filed: Mar. 14, 2007
(86) PCT No.: PCT/EP2007/052415

§ 371 Date: Mar. 3, 2009

(30) Foreign Application Priority Data
Mar. 16, 2006 (GB) 0605414.2
Oct. 27, 2006 (GB) 0621439.9
Feb. 1, 2007 (GB) 0701987.0

(51) Int. Cl. A61K 31/4184; C07D 235/02; A61P 25/00

(52) U.S. Cl. 514/386; 548/300.7

(57) ABSTRACT

Compounds of formula (I) and salts and solvates are provided.

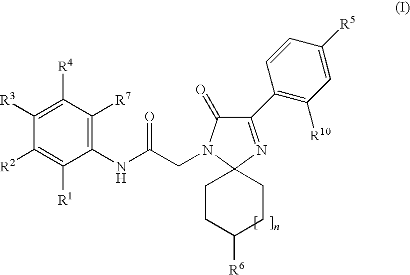

Uses of the compounds as medicaments, and in the manufacture of medicament for treating neurological and neuropsychiatric disorders, in particular psychoses, dementia or attention deficit disorder are also disclosed. The invention further comprises processes to make these compounds and pharmaceutical formulations thereof.

The present invention relates to glycine transporter inhibiting compounds, their use in the manufacture of medicaments for treating neurological and neuropsychiatric disorders, in particular psychoses, dementia or attention deficit disorder. The invention further comprises processes to make these compounds and pharmaceutical formulations thereof.

Molecular cloning has revealed the existence in mammalian brains of two classes of glycine transporters, termed GlyT1 and GlyT2. GlyT1 is found predominantly in the forebrain and its distribution corresponds to that of glutaminergic pathways and NMDA receptors (Smith, et al., Neuron, 8, 1992: 927-935). Molecular cloning has further revealed the existence of three variants of GlyT1, termed GlyT-la, GlyT-1b and GlyT-1c (Kim et al., Molecular Pharmacology, 45, 1994: 608-617), each of which displays a unique distribution in the brain and peripheral tissues. The variants arise by differential splicing and exon usage, and differ in their N-terminal regions. GlyT2, in contrast, is found predominantly in the brain stem and spinal cord, and its distribution corresponds closely to that of strychnine-sensitive glycine receptors (Liu et al., J. Biological Chemistry, 268, 1993: 22802-22808; Jursky and Nelson, J. Neurochemistry, 64, 1995 : 1026-1033). Another distinguishing feature of glycine transport mediated by GlyT2 is that it is not inhibited by sarcosine as is the case for glycine transport mediated by GlyT1. These data are consistent with the view that, by regulating the synaptic levels of glycine, GlyT1 and GlyT2 selectively influence the activity of NMDA receptors and strychnine-sensitive glycine receptors, respectively.

NMDA receptors are critically involved in memory and learning (Rison and Staunton, Neurosci. Biobehav. Rev., 19 533-552 (1995); Danysz et al, Behavioral Pharmacol., 6 455-474 (1995)); and, furthermore, decreased function of NMDA-mediated neurotransmission appears to underlie, or contribute to, the symptoms of schizophrenia (Olney and Farber, Archives General Psychiatry, 52, 998-1007 (1996). Thus, agents that inhibit GlyT1 and thereby increase glycine activation of NMDA receptors can be used as novel antipsychotics and anti-dementia agents, and to treat other diseases in which cognitive processes are impaired, such as attention deficit disorders and organic brain syndromes. Conversely, over-activation of NMDA receptors has been implicated in a number of disease states, in particular the neuronal death associated with stroke and possibly neurodegenerative diseases, such as Alzheimer's disease, multi-infarct dementia, AIDS dementia, Huntington's disease, Parkinson's disease, amyotrophic lateral sclerosis or other conditions in which neuronal cell death occurs, such as stroke or head trauma. Coyle & Puttfarcken, Science 262, 689-695 (1993); Lipton and Rosenberg, New Enql. J. of Medicine, 330, 613-622 (1993); Choi, Neuron, 1, 623-634 (1988). Thus, pharmacological agents that increase the activity of GlyT1 will result in decreased glycine-activation of NMDA receptors, which activity can be used to treat these and related disease states. Similarly, drugs that directly block the glycine site of the NMDA receptors can be used to treat these and related disease states.

Glycine transport inhibitors are already known in the art, for example as disclosed in published international patent application WO03/055478 (SmithKline Beecham).

A novel class of compounds which inhibit GlyT1 transporters have been found. The compounds are of potential use in the treatment of certain neurological and neuropsychiatric disorders, including schizophrenia.

Thus, in the first aspect, there is provided a compound of formula (I) or a salt or solvate thereof:

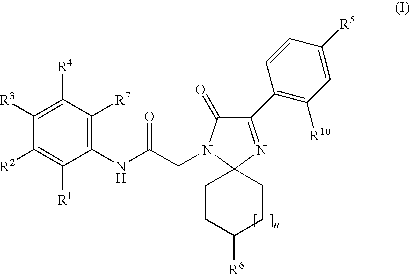

wherein:

R¹ is selected from hydrogen, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl and halo;

R² is selected from hydrogen, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl and halo;

R³is selected from hydrogen, C₁-C₄alkyl, C₁-C₄alkoxy, haloC₁-C₄alkyl, haloC₁-C₄alkoxy, cyano and halo;

or R² and R³ together form a group selected from —O—CH₂—O— and —O—CH₂—CH₂—O—;

R⁵is selected from hydrogen, C₁-C₄alkyl, C₁-C₄alkoxy, chloro and fluoro;

R⁶ is selected from hydrogen and methyl;

R⁷ is selected from hydrogen, chloro, fluoro, C₁-C₄alkyl, CF₃ and CONR⁸R⁹ wherein R⁸ and R⁹ are independently hydrogen and C₁-C₄alkyl, or R⁸ and R⁹, together with the nitrogen atom to which they are attached, form a 4- to 7-membered ring;

n is selected from 0, 1 and 2;

R¹⁰ is selected from hydrogen and fluoro; and

R⁴:
(i) when R¹ is chloro or R⁷ is CONR⁸R⁹, then R⁴is selected from hydrogen, C₁-C₄alkyl, C₁-C₄alkoxy, haloC₁-C₄alkyl, haloC₁-C₄alkoxy, cyano and halo;
(ii) when at least one of R¹, R² and R³ is selected from the group consisting of cyano, haloC₁-C₄alkyl, C₁-C₄alkylsulfonyl, and haloC₁-C₄alkoxy, R⁴ is selected from hydrogen, C₁-C₄alkyl, C₁-C₄alkoxy, haloC₁-C₄alkyl, haloC₁-C₄alkoxy, cyano and halo;
(iii) when simultaneously R¹ is hydrogen, R² is hydrogen or methoxy, and R³ is selected from the group consisting of hydrogen, methyl, ethyl, methoxy, ethoxy, fluoro and chloro, R⁴ is selected from cyano, haloC₁-C₄alkyl, haloC₁-C₄alkoxy and C₁-C₄alkylsulfonyl;
(iv) when R¹ is selected from C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl and halo, and R⁷ is selected from chloro, fluoro, C₁-C₄alkyl, CF₃ and CONR⁸R⁹, then R⁴ is selected from hydrogen, C₁-C₄alkyl, C₁-C₄alkoxy, haloC₁-C₄alkyl, haloC₁-C₄alkoxy, cyano and halo;
(v) in all other cases, R⁴ is selected from methyl, chloro, fluoro, cyano, haloC₁-C₄alkyl, and haloC₁-C₄alkoxy.

The present invention also provides a compound of formula (Ia) or a salt or solvate thereof:

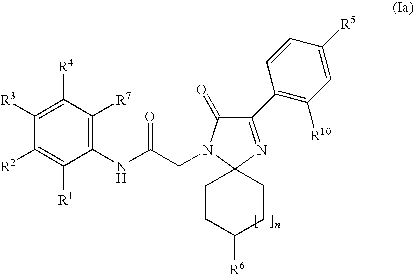

wherein:

R¹ is selected from H, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl and halo;

R² is selected from H, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl and halo;

R³ is selected from H, C₁-C₄alkyl, C₁-C₄alkoxy, haloC₁-C₄alkyl, haloC₁-C₄alkoxy, cyano and halo;

or R² and R³ together form a group selected from —O—CH₂—O— and —O—CH₂—CH₂—O—;

R⁵ is selected from H, C₁-C₄alkyl, C₁-C₄alkoxy, chloro and fluoro;

R⁶ is selected from H and methyl;

R⁷ is selected from hydrogen, chloro, fluoro, C₁-C₄alkyl, CF₃ and CONR⁸R⁹ wherein R⁸ and R⁹ are independently hydrogen and C₁-C₄alkyl, or R⁸ and R⁹, together with the nitrogen atom to which they are attached, form a 4- to 7-membered ring;

n is selected from 0, 1 and 2;

R¹⁰ is selected from hydrogen and fluoro; and

R⁴:

(i) when R¹ is chloro, R⁴ is selected from hydrogen, C₁-C₄alkyl, C₁-C₄alkoxy, haloC₁-C₄alkyl, haloC₁-C₄alkoxy, cyano and halo;

(ii) when at least one of R¹, R² and R³ is selected from the group consisting of cyano, haloC₁-C₄alkyl, C₁-C₄alkylsulfonyl, and haloC₁-C₄alkoxy, R⁴ is selected from hydrogen, C₁-C₄alkyl, C₁-C₄alkoxy, haloC₁-C₄alkyl, haloC₁-C₄alkoxy, cyano and halo;

(iii) when simultaneously R¹ is hydrogen, R² is hydrogen or methoxy, and R³ is selected from the group consisting of hydrogen, methyl, ethyl, methoxy, ethoxy, fluoro and chloro, R⁴ is selected from cyano, haloC₁-C₄alkyl, haloC₁-C₄alkoxy and C₁-C₄alkylsulfonyl;

(iv) when R⁷ is chloro, fluoro, C₁-C₄alkyl, CF₃ or a group CONR⁸R⁹ as defined above, R⁴ is selected from H, C₁-C₄alkyl, C₁-C₄alkoxy, haloC₁-C₄alkyl, haloC₁-C₄alkoxy, cyano and halo;

(v) in all other cases, R⁴ is selected from methyl, chloro, fluoro, cyano, haloC₁-C₄alkyl, and haloC₁-C₄alkoxy;

excluding

N-(2-chlorophenyl)-2-[3-(4-fluorophenyl)-2-oxo-1,4-diazaspiro[4.6]undec-3-en-1-yl]acetamide.

The present invention also provides a compound of formula (Ib) or a salt or solvate thereof:

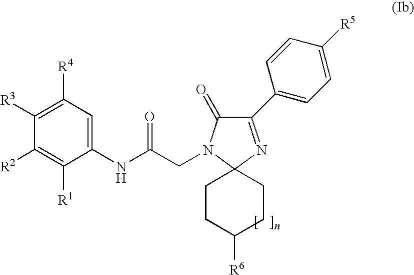

wherein:

R¹ is selected from H, C₁-C₄alkyl, C₁-C₄alkoxy, haloC₁-C₄alkyl, haloC₁-C₄alkoxy, cyano and halo;

R² is selected from H, C₁-C₄alkyl, C₁-C₄alkoxy, haloC₁-C₄alkyl, haloC₁-C₄alkoxy, cyano and halo;

R³ is selected from H, C₁-C₄alkyl, C₁-C₄alkoxy, haloC₁-C₄alkyl, haloC₁-C₄alkoxy, cyano and halo;

or R² and R³ together form a group selected from —O—CH₂—O— and —O—CH₂—CH₂—O—;

R⁵ is selected from H, C₁-C₄alkyl, C₁-C₄alkoxy, chloro and fluoro;

R⁶ is selected from H and methyl;

n is selected from 0, 1 and 2; and

R⁴:

(i) when R¹ is chloro, R⁴ is selected from H, C₁-C₄alkyl, C₁-C₄alkoxy, haloC₁-C₄alkyl, haloC₁-C₄alkoxy, cyano and halo;

(ii) when at least one of R¹, R² and R³ is selected from the group consisting of cyano, haloC₁-C₄alkyl, and haloC₁-C₄alkoxy, R⁴ is selected from H, C₁-C₄alkyl, C₁-C₄alkoxy, haloC₁-C₄alkyl, haloC₁-C₄alkoxy, cyano and halo;

(iii) when simultaneously R¹ is H, R² is H or methoxy, and R³ is selected from the group consisting of H, methyl, ethyl, methoxy, ethoxy, fluoro and chloro, R⁴ is selected from cyano, haloC₁-C₄alkyl, and haloC₁-C₄alkoxy;

(iv) otherwise, R⁴ is selected from methyl, chloro, fluoro, cyano, haloC₁-C₄alkyl, and haloC₁-C₄alkoxy.

The present invention also provides a compound of formula (Ic) or a salt or solvate thereof:

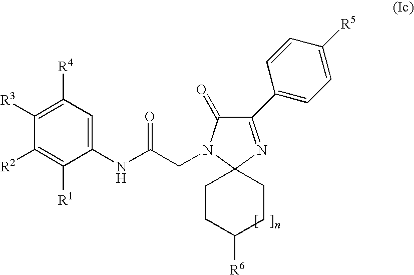

wherein:

R¹ is selected from H, C₁-C₄alkyl, C₁-C₄alkoxy, haloC₁-C₄alkyl, haloC₁-C₄alkoxy, cyano and halo;

R² is selected from H, C₁-C₄alkyl, C₁-C₄alkoxy, haloC₁-C₄alkyl, haloC₁-C₄alkoxy, cyano and halo;

R³ is selected from H, C₁-C₄alkyl, C₁-C₄alkoxy, haloC₁-C₄alkyl, haloC₁-C₄alkoxy, cyano and halo;

or R² and R³ together form a group selected from —O—CH₂—O— and —O—CH₂—CH₂—O—;

R⁵ is selected from H, C₁-C₄alkyl, C₁-C₄alkoxy, chloro and fluoro;

R⁶ is selected from H and methyl;

n is selected from 0, 1 and 2; and

R⁴:

(i) when R¹ is chloro, R⁴ is selected from H, C₁-C₄alkyl, C₁-C₄alkoxy, haloC₁-C₄alkyl, haloC₁-C₄alkoxy, cyano and halo;

(ii) when at least one of R¹, R² and R³ is selected from the group consisting of cyano, haloC₁-C₄alkyl, and haloC₁-C₄alkoxy, R⁴ is selected from H, C₁-C₄alkyl, C₁-C₄alkoxy, haloC₁-C₄alkyl, haloC₁-C₄alkoxy, cyano and halo;

(iii) when simultaneously R¹ is H, R² is H or methoxy, and R³ is selected from the group consisting of H, methyl, ethyl, methoxy, ethoxy, fluoro and chloro, R⁴ is selected from cyano, haloC₁-C₄alkyl, and haloC₁-C₄alkoxy;

(iv) otherwise, R⁴ is selected from methyl, chloro, fluoro, cyano, haloC₁-C₄alkyl, and haloC₁-C₄alkoxy;

excluding

N-(2-chlorophenyl)-2-[3-(4-fluorophenyl)-2-oxo-1,4-diazaspiro[4.6]undec-3-en-1-yl]acetamide.

As used herein, the term “C₁-C₄alkyl” refers to a straight or branched alkyl group in all isomeric forms. Examples include methyl, ethyl, propyl, isopropyl, butyl, isobutyl, sec-butyl and tert-butyl.

As used herein, the term “alkoxy” refers to the group —O-alkyl wherein alkyl is as defined above.

As used herein, the terms “halogen” and its abbreviation “hal” refer to fluorine, chlorine, bromine, or iodine.

As used herein, the term “haloC₁-C₄alkyl” refers to a C₁-C₄alkyl group as defined above which is substituted with any number of fluorine, chlorine, bromine, or iodine atoms, including with mixtures of those atoms. A haloalkyl group may, for example contain 1, 2 or 3 halogen atoms. For example, a haloalkyl group may have all hydrogen atoms replaced with halogen atoms. Examples of haloC₁-C₄alkyl groups include fluoromethyl, difluoromethyl and trifluoromethyl.

As used herein, the term “haloC₁-C₄alkoxy” refers to a C₁-C₄alkoxy group as defined above which is substituted with any number of fluorine, chlorine, bromine, or iodine atoms, including with mixtures of those atoms. A haloalkoxy group may, for example contain 1, 2 or 3 halogen atoms. For example, a haloalkoxy group may have all hydrogen atoms replaced with halogen atoms. Examples of haloC₁-C₄alkoxy groups include fluoromethyloxy, difluoromethyloxy and trifluoromethyloxy.

As used herein, the term “C₁-C₄alkylsulfonyl” refers to a group —SO₂(C₁-C₄alkyl). An example is methylsulfonyl (—SO₂CH₃). Similarly, the term “haloC₁-C₄alkylsulfonyl” refers to a group —SO₂(haloC₁-C₄alkyl). An example is trifluromethylsulfonyl (—SO₂CF₃).

The term “4- to 7-membered ring” as used in the definition of R⁸ and R⁹ refers to a 4, 5, 6 or 7-membered saturated ring formed by R⁸ and R⁹ and the nitrogen atom to which they are attached. Examples include azetidinyl, pyrrolidinyl, piperidinyl and azepanyl.

In one embodiment, n is 0 or 1. In one embodiment, n is 1.

In one embodiment, R⁶ is hydrogen.

The present invention also provides a compound of formula (Id) or a salt or solvate thereof:

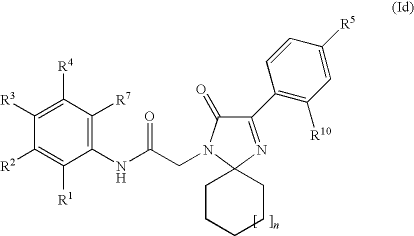

wherein:

R¹ is selected from hydrogen, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl and halo;

R² is selected from hydrogen, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl and halo;

R³is selected from hydrogen, C₁-C₄alkyl, C₁-C₄alkoxy, haloC₁-C₄alkyl, haloC₁-C₄alkoxy, cyano and halo;

or R² and R³ together form a group selected from —O—CH₂—O— and —O—CH₂—CH₂—O—;

R⁵is selected from hydrogen, C₁-C₄alkyl, C₁-C₄alkoxy, chloro and fluoro;

R⁷ is selected from hydrogen, chloro, fluoro, C₁-C₄alkyl, CF₃ and CONR⁸R⁹ wherein R⁸ and R⁹ are independently hydrogen and C₁-C₄alkyl, or R⁸ and R⁹, together with the nitrogen atom to which they are attached, form a 4- to 7-membered ring;

n is selected from 0, 1 and 2;

R¹⁰ is selected from hydrogen and fluoro; and

R⁴:
(i) when R¹ is chloro or R⁷is CONR⁸R⁹, then R⁴ is selected from hydrogen, C₁-C₄alkyl, C₁-C₄alkoxy, haloC₁-C₄alkyl, haloC₁-C₄alkoxy, cyano and halo;
(ii) when at least one of R¹, R² and R³ is selected from the group consisting of cyano, haloC₁-C₄alkyl, C₁-C₄alkylsulfonyl, and haloC₁-C₄alkoxy, R⁴ is selected from hydrogen, C₁-C₄alkyl, C₁-C₄alkoxy, haloC₁-C₄alkyl, haloC₁-C₄alkoxy, cyano and halo;
(iii) when simultaneously R¹ is hydrogen, R² is hydrogen or methoxy, and R³ is selected from the group consisting of hydrogen, methyl, ethyl, methoxy, ethoxy, fluoro and chloro, R⁴ is selected from cyano, haloC₁-C₄alkyl, haloC₁-C₄alkoxy and C₁-C₄alkylsulfonyl;
(iv) when R¹ is selected from C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl and halo, and R⁷ is selected from chloro, fluoro, C₁-C₄alkyl, CF₃ and CONR⁸R⁹, then R⁴ is selected from hydrogen, C₁-C₄alkyl, C₁-C₄alkoxy, haloC₁-C₄alkyl, haloC₁-C₄alkoxy, cyano and halo;
(v) in all other cases, R⁴is selected from methyl, chloro, fluoro, cyano, haloC₁-C₄alkyl, and haloC₁-C₄alkoxy.

The present invention also provides a compound of formula (Ie) or a salt or solvate thereof:

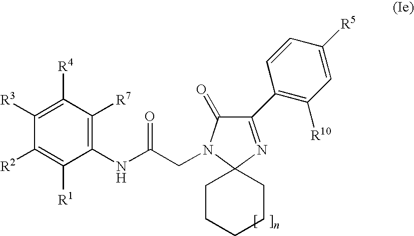

wherein:

R¹ is selected from hydrogen, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl and halo;

R² is selected from hydrogen, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl and halo;

R³is selected from hydrogen, C₁-C₄alkyl, C₁-C₄alkoxy, haloC₁-C₄alkyl, haloC₁-C₄alkoxy, cyano and halo;

or R² and R³ together form a group selected from —O—CH₂—O— and —O—CH₂—CH₂—O—;

R⁵is selected from hydrogen, C₁-C₄alkyl, C₁-C₄alkoxy, chloro and fluoro;

R⁷ is selected from hydrogen, chloro, fluoro, C₁-C₄alkyl, CF₃ and CONR⁸R⁹ wherein R⁸ and R⁹ are independently hydrogen and C₁-C₄alkyl, or R⁸ and R⁹, together with the nitrogen atom to which they are attached, form a 4- to 7-membered ring;

n is selected from 0, 1 and 2;

R¹⁰ is selected from hydrogen and fluoro; and

R⁴:
(i) when R¹ is chloro or R⁷is CONR⁸R⁹, then R⁴ is selected from hydrogen, C₁-C₄alkyl, C₁-C₄alkoxy, haloC₁-C₄alkyl, haloC₁-C₄alkoxy, cyano and halo;
(ii) when at least one of R¹, R² and R³ is selected from the group consisting of cyano, haloC₁-C₄alkyl, C₁-C₄alkylsulfonyl, and haloC₁-C₄alkoxy, R⁴ is selected from hydrogen, C₁-C₄alkyl, C₁-C₄alkoxy, haloC₁-C₄alkyl, haloC₁-C₄alkoxy, cyano and halo;
(iii) when simultaneously R¹ is hydrogen, R² is hydrogen or methoxy, and R³ is selected from the group consisting of hydrogen, methyl, ethyl, methoxy, ethoxy, fluoro and chloro, R⁴ is selected from cyano, haloC₁-C₄alkyl, haloC₁-C₄alkoxy and C₁-C₄alkylsulfonyl;
(iv) when R¹ is selected from C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl and halo, and R⁷ is selected from chloro, fluoro, C₁-C₄alkyl, CF₃ and CONR⁸R⁹, then R⁴ is selected from hydrogen, C₁-C₄alkyl, C₁-C₄alkoxy, haloC₁-C₄alkyl, haloC₁-C₄alkoxy, cyano and halo;
(v) in all other cases, R⁴ is selected from methyl, chloro, fluoro, cyano, haloC₁-C₄alkyl, and haloC₁-C₄alkoxy;

excluding N-(2-chlorophenyl)-2-[3-(4-fluorophenyl)-2-oxo-1,4-diazaspiro[4.6]undec-3-en-1-yl]acetamide.

In one embodiment, R¹ is selected from hydrogen, methyl, methoxy, trifluoromethyl, trifluoromethoxy, cyano, methylsulfonyl, trifluoromethylsulfonyl and halo. In one embodiment, R¹ is selected from hydrogen, methyl, methoxy, methylsulfonyl, cyano and halo. In one embodiment, R¹ is selected from hydrogen, cyano, chloro, fluoro and methylsulfonyl. In one embodiment, R¹ is selected from hydrogen, cyano, chloro and fluoro.

In one embodiment, R² is selected from hydrogen, methyl, methoxy, trifluoromethyl, trifluoromethoxy, cyano, methylsulfonyl, trifluoromethylsulfonyl and halo. In one embodiment, R² is selected from hydrogen, methyl, methylsulfonyl, trifluoromethyl, cyano and halo. In one embodiment, R² is selected from hydrogen, methyl, trifluoromethyl, cyano, methylsulfonyl, chloro, fluoro and bromo. In one embodiment, R² is selected from hydrogen, trifluoromethyl, cyano, chloro and fluoro.

In one embodiment, R³ is selected from hydrogen, methyl, methoxy, ethoxy, trifluoromethyl, trifluoromethoxy, cyano and halo; or R² and R³ together form a group selected from —O—CH₂—O— and —O—CH₂—CH₂—O—. In one embodiment, R³ is selected from hydrogen, methyl, methoxy, cyano and halo. In one embodiment, R³ is selected from hydrogen, cyano, and fluoro.

In one embodiment, R⁷ is selected from hydrogen, fluoro and CONR⁸R⁹ wherein R⁸ and R⁹ are independently hydrogen and C₁-C₄alkyl. In one embodiment, R⁷ is selected from hydrogen, fluoro and CONH₂.

In one embodiment, R¹⁰ is hydrogen.

In one embodiment:

(i) when R¹ is chloro, R⁴ is selected from hydrogen, methyl, methoxy, trifluoromethyl, trifluoromethoxy, cyano and halo;

(ii) when at least one of R¹, R² and R³ is selected from the group consisting of cyano, haloC₁-C₄alkyl, C₁-C₄alkylsulfonyl, and haloC₁-C₄alkoxy, R⁴ is selected from hydrogen, methyl, methoxy, trifluoromethyl, trifluoromethoxy, cyano and halo;

(iii) when simultaneously R¹ is hydrogen, R² is hydrogen or methoxy, and R³ is selected from the group consisting of hydrogen, methyl, ethyl, methoxy, ethoxy, fluoro and chloro, R⁴ is selected from cyano, trifluoromethyl, trifluoromethoxy and methylsulfonyl;

(iv) when R¹ is selected from C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl and halo, and R⁷is selected from chloro, fluoro, C₁-C₄alkyl, CF₃ and CONR⁸R⁹, then R⁴ is selected from hydrogen, methyl, methoxy, trifluoromethyl, trifluoromethoxy, cyano and halo;

(v) in all other cases, R⁴ is selected from methyl, chloro, fluoro, cyano, trifluoromethyl, and trifluoromethoxy.

In one embodiment, (i) applies. In another embodiment, (ii) applies. In a further embodiment (iii) applies. In a still further embodiment, (iv) applies. In a further embodiment, (v) applies.

In one embodiment, when (i) applies, R⁴ is hydrogen.

In one embodiment, when (ii) applies, R⁴ is hydrogen or haloC₁-C₄alkyl. In one embodiment, when (ii) applies, R⁴ is hydrogen or trifluoromethyl.

In one embodiment, when (iii) applies, R⁴ is selected from haloC₁-C₄alkyl and C₁-C₄alkylsulfonyl. In one embodiment, (iii) applies, R⁴ is selected from trifluoromethyl, and methylsulfonyl.

In one embodiment, when (iv) applies, R⁴ is selected from hydrogen, methyl, methoxy, trifluoromethyl, trifluoromethoxy, cyano and halo. In one embodiment, when (iv) applies, R⁴ is hydrogen.

In one embodiment, when (v) applies, R⁴ is selected from fluoro, cyano, haloC₁-C₄alkyl, and haloC₁-C₄alkoxy. In one embodiment when (v) applies, R⁴ is selected from cyano, haloC₁-C₄alkyl, and haloC₁-C₄alkoxy. In one embodiment, when (v) applies, R⁴ is trifluoromethyl.

In one embodiment, R⁴ is selected from cyano, haloC₁-C₄alkyl, and haloC₁-C₄alkoxy.

In one embodiment, R⁵ is selected from hydrogen, methyl, methoxy, chloro and fluoro. In one embodiment, R⁵ is selected from methyl, methoxy and chloro. In one embodiment, R⁵ is selected from methoxy and chloro.

For the avoidance of doubt, the embodiments of any one feature of the compounds of the invention may be combined with any embodiment of another feature of compounds of the invention to create a further embodiment.

As used herein, the term “salt” refers to any salt of a compound according to the present invention prepared from an inorganic or organic acid or base, quaternary ammonium salts and internally formed salts. Pharmaceutically acceptable salts are particularly suitable for medical applications because of their greater aqueous solubility relative to the parent compounds. Such salts must clearly have a pharmaceutically acceptable anion or cation. Suitably pharmaceutically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, (1S)-(−)-10-camphorsulphonic, (1S)-(+)-10-camphorsulphonic, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example naphthalene-1,5-disulphonic, naphthalene-1,3-disulphonic, benzenesulfonic, and p-toluenesulfonic, acids; base addition salts formed with alkali metals and alkaline earth metals and organic bases such as N,N-dibenzylethylenediamine, chloroprocaine, choline, diethanolamine, ethylenediamine, meglumaine (N-methylglucamine), lysine and procaine; and internally formed salts. Salts having a non-pharmaceutically acceptable anion or cation are within the scope of the invention as useful intermediates for the preparation of pharmaceutically acceptable salts and/or for use in non-therapeutic, for example, in vitro, situations. The salts may have any suitable stoichiometry. For example, a salt may have 1:1 or 2:1 stoichiometry. Non-integral stoichiometry ratios are also possible.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include, but are not limited to, water, methanol, ethanol and acetic acid. In one embodiment, the solvent used is a pharmaceutically acceptable solvent. Examples of suitable pharmaceutically acceptable solvents include water, ethanol and acetic acid. In one embodiment, the solvent used is water.

Examples of compounds of the invention include:

2-[3-(4-Chlorophenyl)-2-oxo-1,4-diazaspiro[4.5]dec-3-en-1-yl]-N-(3,5-difluorophenyl)acetamide;

2-{3-[4-(Methyloxy)phenyl]-2-oxo-1,4-diazaspiro[4.5]dec-3-en-1-yl}-N-[3-(trifluoromethyl)phenyl]acetamide;

2-[3-(4-Chlorophenyl)-2-oxo-1,4-diazaspiro[4.5]dec-3-en-1-yl]-N-(2-cyanophenyl)acetamide;

2-[3-(4-chlorophenyl)-2-oxo-1,4-diazaspiro[4.5]dec-3-en-1-yl]-N-(3-cyanophenyl)acetamide;

2-[3-(4-chlorophenyl)-2-oxo-1,4-diazaspiro[4.5]dec-3-en-1-yl]-N-(4-cyanophenyl)acetamide;

2-[3-(4-chlorophenyl)-2-oxo-1,4-diazaspiro[4.5]dec-3-en-1-yl]-N-[2-chloro-3-(trifluoromethyl)phenyl]acetamide;

2-[3-(4-chlorophenyl)-2-oxo-1,4-diazaspiro[4.5]dec-3-en-1-yl]-N-[3-(trifluoromethyl)phenyl]acetamide;

2-[3-(4-chlorophenyl)-2-oxo-1,4-diazaspiro[4.5]dec-3-en-1-yl]-N-(2,3-dichlorophenyl)acetamide;

N-(3-cyanophenyl)-2-{3-[4-(methyloxy)phenyl]-2-oxo-1,4-diazaspiro[4.5]dec-3-en-1-yl}acetamide;

2-[3-(4-chlorophenyl)-2-oxo-1,4-diazaspiro[4.5]dec-3-en-1-yl]-N-(2,5-difluorophenyl)acetamide;

and salts and solvates thereof.

Other examples of compounds of the invention include:

N-(3,5-Difluorophenyl)-2-{3-[4-(methyloxy)phenyl]-2-oxo-1,4-diazaspiro[4.5]dec-3-en-1-yl}acetamide

N-(3,5-Difluorophenyl)-2-{3-[4-(methyloxy)phenyl]-2-oxo-1,4-diazaspiro[4.4]non-3-en-1-yl}acetamide

N-[3,5-bis(trifluoromethyl)phenyl]-2-{3-[4-(methyloxy)phenyl]-2-oxo-1,4-diazaspiro[4.5]dec-3-en-1-yl}acetamide

N-[3-cyano-5-(trifluoromethyl)phenyl]-2-{3-[4-(methyloxy)phenyl]-2-oxo-1,4-diazaspiro[4.5]dec-3-en-1-yl}acetamide

N-[3-bromo-5-(trifluoromethyl)phenyl]-2-{3-[4-(methyloxy)phenyl]-2-oxo-1,4-diazaspiro[4.5]dec-3-en-1-yl}acetamide

N-(2-fluoro-5-methylphenyl)-2-{3-[4-(methyloxy)phenyl]-2-oxo-1,4-diazaspiro[4.5]dec-3-en-1-yl}acetamide

2-{3-[4-(methyloxy)phenyl]-2-oxo-1,4-diazaspiro[4.5]dec-3-en-1-yl}-N-(2,4,5-trifluorophenyl)acetamide

2-{3-[4-(methyloxy)phenyl]-2-oxo-1,4-diazaspiro[4.5]dec-3-en-1-yl}-N-(2,3,5-trifluorophenyl)acetamide

2-{3-[4-(methyloxy)phenyl]-2-oxo-1,4-diazaspiro[4.5]dec-3-en-1-yl}-N-(3,4,5-trifluorophenyl)acetamide

N-(2-chlorophenyl)-2-[3-(4-fluorophenyl)-2-oxo-1,4-diazaspiro[4.6]undec-3-en-1-yl]acetamide

and salts and solvates thereof.

Other examples of the invention include:

2-[3-(4-chlorophenyl)-2-oxo-1,4-diazaspiro[4.5]dec-3-en-1-yl]-N-(2,6-difluorophenyl)acetamide

2-{3-[4-(methyloxy)phenyl]-2-oxo-1,4-diazaspiro[4.5]dec-3-en-1-yl}-N-(2,3,6-trifluorophenyl)acetamide

N-(3-fluoro-5-methylphenyl)-2-{3-[4-(methyloxy)phenyl]-2-oxo-1,4-diazaspiro[4.5]dec-3-en-1-yl}acetamide

2-{3-[4-(methyloxy)phenyl]-2-oxo-1,4-diazaspiro[4.5]dec-3-en-1-yl}-N-(2,3,5-trifluorophenyl)acetamide

2-[3-(4-chlorophenyl)-2-oxo-1,4-diazaspiro[4.5]dec-3-en-1-yl]-N-[2-(methylsulfonyl)phenyl]acetamide

2-[3-(4-chlorophenyl)-2-oxo-1,4-diazaspiro[4.5]dec-3-en-1-yl]-N-[3-(methylsulfonyl)phenyl]acetamide

2-({[3-(4-chlorophenyl)-2-oxo-1,4-diazaspiro[4.5]dec-3-en-1-yl]acetyl}amino)benzamide

and salts and solvates thereof.

Other examples of the invention include:

2-[3-(4-chlorophenyl)-2-oxo-1,4-diazaspiro[4.4]non-3-en-1-yl]-N-(3-cyano-4-methylphenyl)acetamide

2-[3-(4-chlorophenyl)-2-oxo-1,4-diazaspiro[4.4]non-3-en-1-yl]-N-(3-cyano-5-fluorophenyl)acetamide

2-[3-(4-chlorophenyl)-2-oxo-1,4-diazaspiro[4.4]non-3-en-1-yl]-N-(3-cyano-4-fluorophenyl)acetamide

2-[3-(4-chlorophenyl)-2-oxo-1,4-diazaspiro[4.5]dec-3-en-1-yl]-N-(3-cyano-4-fluorophenyl)acetamide

2-[3-(4-chlorophenyl)-2-oxo-1,4-diazaspiro[4.5]dec-3-en-1-yl]-N-(3-cyano-4-methylphenyl)acetamide

2-[3-(4-chlorophenyl)-2-oxo-1,4-diazaspiro[4.5]dec-3-en-1-yl]-N-(3-cyano-5-fluorophenyl)acetamide

and salts and solvates thereof.

The compounds of formula (I) may have the ability to crystallise in more than one form. This is a characteristic known as polymorphism, and it is understood that such polymorphic forms (“polymorphs”) are within the scope of formula (I). Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

Certain of the compounds described herein may exist in stereoisomeric forms (i.e. they may contain one or more asymmetric carbon atoms or may exhibit cis-trans isomerism). The individual stereoisomers (enantiomers and diastereoisomers) and mixtures of these are included within the scope of the present invention. Likewise, it is understood that compounds of formula (I) may exist in tautomeric forms other than that shown in the formula and these are also included within the scope of the present invention.

In one embodiment, an optically pure enantiomer of a compound of the present invention is provided. The term “optically pure enantiomer” means that the compound contains greater than about 90% of the desired isomer by weight, such as greater than about 95% of the desired isomer by weight, or greater than about 99% of the desired isomer by weight, said weight percent based upon the total weight of the isomer(s) of the compound.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the working Examples.

Compounds of general formula (I) may be prepared by methods known in the art of organic synthesis as set forth in part by the following synthesis schemes. It is also recognised that in all of the schemes described below, it is well understood that protecting groups for sensitive or reactive groups are employed where necessary in accordance with general principles of chemistry. Protecting groups are manipulated according to standard methods of organic synthesis (T. W. Greene and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognise if a stereocentre exists in compounds of formula (I). Accordingly, the present invention includes both possible stereoisomers and includes not only racemic compounds but the individual enantiomers as well. Where the stereochemistry is indicated as being variable at certain positions, a mixture of stereoisomers may be obtained, this mixture having been separated where indicated. Stereoisomers may be separated by high-performance liquid chromatography or other appropriate means. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

In the following processes, the substituents have the same meanings as for formula (I) unless otherwise stated.

In another aspect, the present invention provides a process for the manufacture of a compound of formula (I) as defined above, the process comprising:

(a) reacting a compound of formula (II)

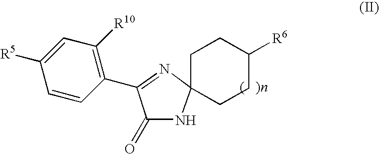

wherein R⁵, R⁶, R¹⁰ and n are as defined for formula (I), with a compound of formula (III):

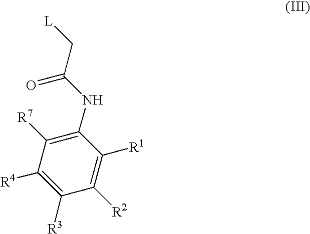

wherein R¹, R², R³, R⁴ and R⁷ are as defined for formula (I), and L is a leaving group;

or

(b) reacting a compound of formula (XV):

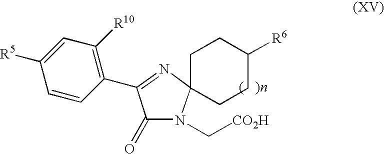

wherein R⁵, R⁶, R¹⁰ and n are as defined for formula (I), with a compound of formula (XVI):

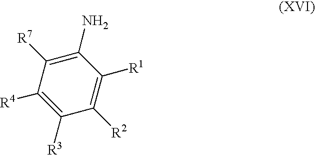

wherein R¹, R², R³, R⁴ and R⁷ are as defined for formula (I);

or

(c) reacting a compound of formula (XVII):

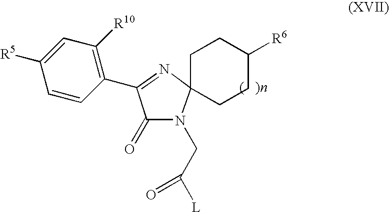

wherein R⁵, R⁶ and R¹⁰ are as defined in formula (I) and L represents a leaving group, with a compound of formula (XVI) as defined in process (b);

and thereafter optionally:
removing any protecting groups and/or
converting a compound of formula (I) into another compound of formula (I) and/or
forming a salt or solvate.

For process (a), a compound for formula (II) may be reacted with a base, for example sodium hydride, in a suitable inert solvent, for example dimethylformamide, followed by treatment with a compound of formula (III).

For process (b), compounds of formula (XV) can be converted to compounds of formula (I), step (vi), by reaction with an aniline of formula (XVI) using a variety of methods known in the art. For example, the acylation step (vi) can be achieved by reaction of the acid (XV) with an aniline of formula (XVI), in an inert solvent, such as dichloromethane in the presence of a coupling reagent, for example a diimide reagent such as N,N dicyclohexylcarbodiimide (DCC), N-(3-(dimethylamino)propyl)-N-ethylcarbodiimide hydrochloride (EDC), or O-(7-azabenzotriazol-1-yl)-1,1,3,3-tetramethyluronium hexafluoro phosphate (HATU).

For process (c), examples of L include halogen, OC(═O)alkyl, OC(═O)O-alkyl and OSO₂Me. In one embodiment, L is halogen and the process is carried out in an inert solvent such as dichloromethane, in the presence of a base, such as triethylamine.

Compounds of formula (III) can be prepared by standard methods, for example as shown in Scheme 2. For example an aniline of formula (IV) may be combined with chloroacetyl chloride in an inert solvent, for example dioxan, and heated to give a compound of formula (III).

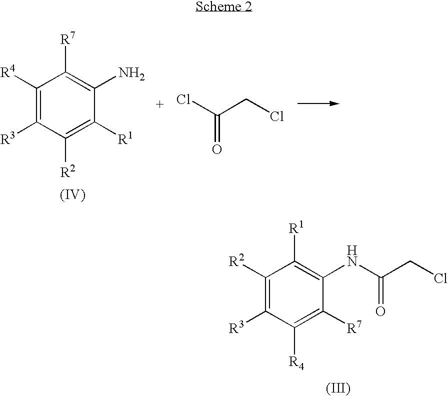

Compounds of formula (II) may be prepared by desulphurisation of compounds of formula (V) using an oxidising agent, for example hydrogen peroxide as shown for example in Scheme 3.

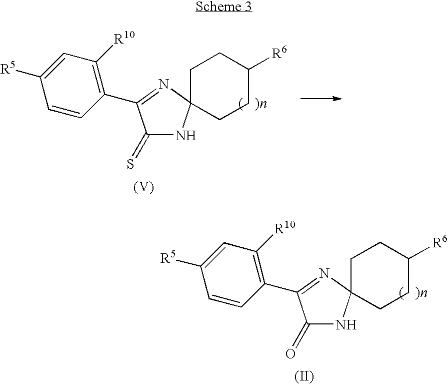

Compounds of formula (V) can be prepared by treating a ketothioamide of formula (VI) with the appropriate ketone of formula (VII) in the presence of a source of ammonia, for example ammonium acetate as shown in Scheme 4. In one embodiment, this reaction is performed in a solvent, for example isopropanol, at room or elevated temperature, preferably elevated temperature, for example at reflux.

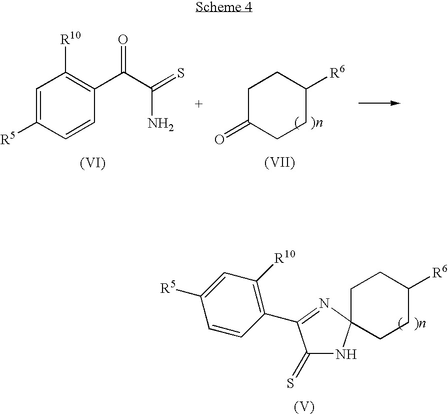

Thioamides of formula (VI) can be prepared from acylnitriles of formula (VIII) by treating with, for example hydrogen sulphide in the presence of an organic base, for example triethylamine in an inert solvent, for example diethyl ether at room temperature. Acylnitriles of formula (VIII) can be prepared from the appropriate acid chloride (IX) and a source of cyanide, conveniently copper (I) cyanide, at elevated temperatures, for example greater than 150° C. preferably in the absence of solvent.

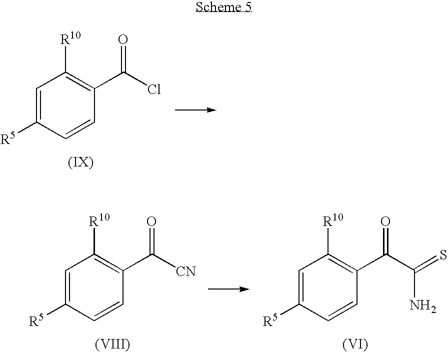

Alternatively, compounds of formula (II) can be synthesised as shown in Scheme 6.

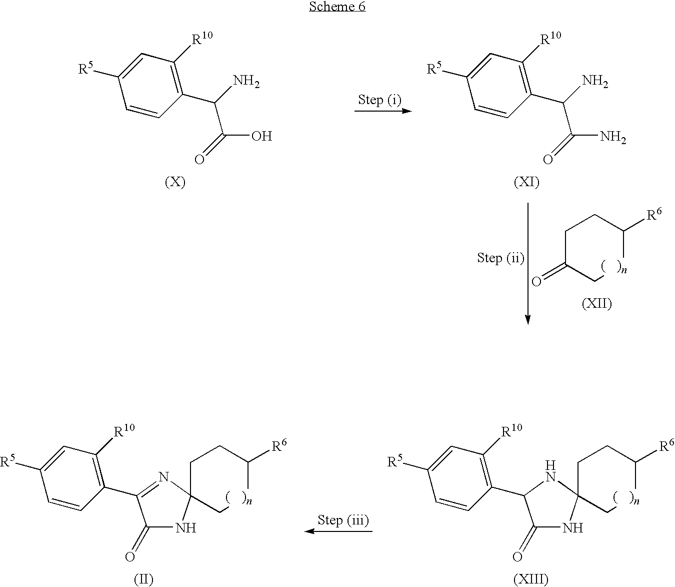

The arylglycine of formula (X) can be converted, step (i), to the corresponding arylglycinamide of formula (XI) by standard methods, for example, by reaction of compounds of formula (X) with thionyl chloride or acetyl chloride in methanol, followed by subsequent reaction of the intermediate methyl ester hydrochloride with aqueous ammonia.

Arylglycinamides of formula (XI) can be converted to compounds of formula (XIII), step (ii), by condensation with ketones of formula (XII), for example, by heating in an inert solvent such as methanol, in the presence or absence of a catalyst such as H—Y zeolites.

Oxidation of compounds of formula (XIII), step (iii), to afford compounds of formula (II) can be achieved by methods known in the art, for example, by reaction with N-bromosuccinimide in an inert solvent, such as dichloromethane.

Compounds of formula (XV) may be prepared as shown in Scheme 7.

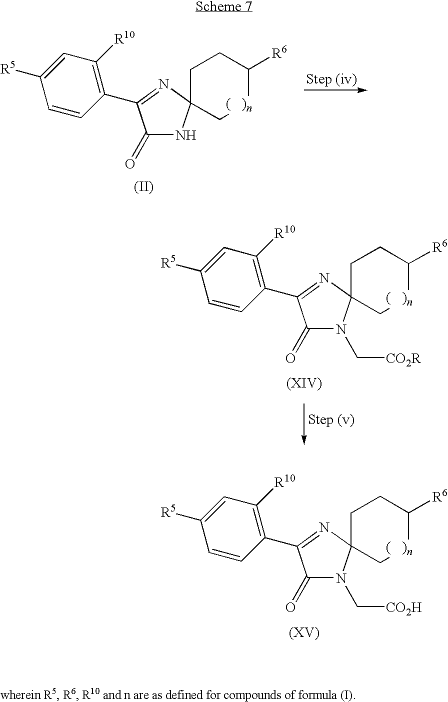

Compounds of formula (XIV) can be prepared using standard methods from compounds of formula (II), step (iv), for example, by reaction with an appropriate haloester in the presence of a base, such as sodium hydride or potassium carbonate, in a suitable inert solvent, such as dimethylformamide, at room temperature or elevated temperature as appropriate.

Removal of the ester group R from compounds of formula (XIV) to afford the acids of formula (XV), step (v), can be achieved by known methods, for example by use of a base, such as sodium hydroxide, in an inert solvent, such as aqueous methanol or aqueous ethanol, with or without heating as appropriate.

Alternatively, compounds of formula (XV) may be converted to compounds of formula (XVII).

Compounds of formula (I) can be converted into further compounds of formula (I) using standard techniques. For example, a group R¹ may be converted into another group R¹ and similarly groups R², R³, R⁴, R⁵, R⁶ and R⁷, using conventional chemistry. Salts may be prepared conventionally by reaction with the appropriate acid or acid derivative.

The compounds of the present invention inhibit the GlyT1 transporter. The compounds may selectively inhibit the GlyT1 transporter over the GlyT2 transporter. Some compounds of the invention may have mixed GlyT-1/GlyT-2 activity.

Such compounds would be suitable for the treatment of certain neurological and neuropsychiatric disorders. As used herein, the terms “treatment” and “treating” refer to the alleviation and/or cure of established symptoms as well as prophylaxis.

The affinities of the compounds of this invention for the GlyT1 transporter can be determined by the following assays.

1) HEK293 cells expressing the Glycine (Type 1) transporter were grown in cell culture medium [DMEM/NUT mix F12 containing 2 mM L-Glutamine, 0.8 mg/mL G418 and 10% heat inactivated fetal calf serum] at 37° C. and 5% CO₂. Cells grown to 70-80% confluency in T175 flasks were harvested and resuspended at 4×10⁵ cells/mL in assay buffer [140 mM NaCl, 5.4 mM KCl, 1.8 mM CaCl₂, 0.8 mM MgSO₄, 20 mM HEPES, 5 mM glucose and 5 mM alanine, pH 7.4]. Compounds were serially diluted 2.5-fold in DMSO from a top concentration of 2.5 mM with each compound giving a 11 data point dose-response. 100 nL of compound at each concentration was added to the assay plate. An equal volume of Leadseeker™ WGA SPA beads (12.5 mg/ml suspended in assay buffer) was added to the cell suspension and 5 μL of the cell/bead suspension transferred to each well of a 384-well white solid bottom plate (1,000 cells/well) containing 100 nL of test compounds. Substrate (5 uL) was added to each well [1:100 dilution of [³H]-glycine stock in assay buffer containing 2.5 uM glycine). Final DMSO concentration was 1% v/v. Data was collected using a Perkin Elmer Viewlux. pIC₅₀ values were determined using ActivityBase.

2) HEK293 cells expressing the Glycine (Type 1) transporter were grown in cell culture medium [DMEM/NUT mix F12 containing 2 mM L-Glutamine, 0.8 mg/mL G418 and 10% heat inactivated fetal calf serum] at 37 C and 5% CO2. Cells grown to 70-80% confluency in T175 flasks were harvested and frozen. For the assay, cells were defrosted and resuspended at 1.32×106 cells/mL in assay buffer [140 mM NaCl, 5.4 mM KCl, 1.8 mM CaCl2, 0.8 mM MgSO4, 20 mM HEPES, 5 mM glucose and 5 mM alanine, pH 7.4]. Compounds were serially diluted 4-fold in DMSO from a top concentration of 2.5 mM with each compound giving a 11 data point dose-response. 100 nL of compound at each concentration was added to the assay plate. An equal volume of Leadseeker™ WGA SPA beads (12.5 mg/ml suspended in assay buffer) was added to the cell suspension (1.32×106) and 5 uL of the cell/bead suspension transferred to each well of a LV384-well white solid bottom plate (3300 cells/well) containing 100 nL of test compounds. Substrate (5 uL) was added to each well [1:100 dilution of [3H]-glycine stock in assay buffer containing 2.5 uM glycine). Final DMSO concentration was 1% v/v. Data was collected using a Perkin Elmer Viewlux. pIC50 values were determined using ActivityBase.

Compounds are considered to have activity at the the GlyT1 transporter if they have a pIC₅₀ of 5.0 or above. The example compounds below and the individually named compounds above were found to have a pIC₅₀ at the GlyT1 transporter of 5.9 or above. Some compounds of the invention were found to have a pIC₅₀ at the GlyT1 transporter of greater than 7.0.

In one aspect of the invention, there is provided a compound of formula (I) or a salt or solvate thereof, as defined in the first aspect of the present invention, for use as a medicament.

In one aspect of the invention, there is provided a compound of formula (I) or a salt or solvate thereof, as defined in the first aspect of the present invention, for use in the treatment of a disorder mediated by GlyT1.

Hereinafter, all references to compounds of formula (I) and salts and solvate thereof are intended to include compounds of formula (Ia), (Ib), (Ic), (Id) and (Ie) and salts and solvates thereof.

In order to use a compound of the present invention as a medicament, it will normally be formulated into a pharmaceutical composition in accordance with standard pharmaceutical practice. The present invention also provides a pharmaceutical composition, which comprises a compound of formula (I) or a salt or solvate thereof, and a carrier, diluent or excipient.

In a further aspect, the present invention provides a process for preparing a pharmaceutical composition, the process comprising mixing a compound of formula (I) or a salt or solvate thereof and a carrier, diluent or excipient.

As used herein, the term “a disorder mediated by GlyT1” refers to a disorder that may be treated by the administration of a medicament that alters the activity of the GlyT1 transporter. As hereinbefore described, the action of GlyT1 transporters affects the local concentration of glycine around NMDA receptors. As a certain amount of glycine is needed for the efficient functioning of NMDA receptors, any change to that local concentration can affect NMDA-mediated neurotransmission. As hereinbefore described, changes in NMDA-mediated neurotransmission have been implicated in certain neuropsychiatric disorders such as dementia, depression and psychoses, for example schizophrenia, and learning and memory disorders, for example attention deficit disorders and autism. Thus, alterations in the activity of the GlyT1 transporter are expected to influence such disorders.

The disorders mediated by GlyT1 referred to herein include neurological and neuropsychiatric disorders, including psychoses such as schizophrenia, dementia and other forms of impaired cognition such as attention deficit disorders and organic brain syndromes. Other neuropsychiatric disorders include drug-induced (phencyclidine, ketamine and other dissociative anesthetics, amphetamine and other psychostimulants and cocaine) psychosis, psychosis associated with affective disorders, brief reactive psychosis, schizoaffective psychosis, and psychosis NOS, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), and NMDA receptor-related disorders such as autism, depression, benign forgetfulness, childhood learning disorders and closed head injury. Other disorders include Parkinson's disease, dyskinetic disorders, cognitive impairment, emesis, movement disorders, amnesia, circadian rhythm disorders, aggression and vertigo.

In another aspect of the invention, there is provided a method of treating a mammal, including a human, suffering from or susceptible to a disorder mediated by GlyT1, which comprises administering an effective amount of a compound of formula (I) as hereinbefore defined or a salt or solvate thereof.

In another aspect of the invention, there is provided use of a compound of formula (I) as hereinbefore defined or a salt or solvate thereof in the preparation of a medicament for the treatment of a disorder mediated by GlyT1.

In one embodiment, the disorder mediated by GlyT1 to be treated by the use or method as hereinbefore described is a psychosis, including schizophrenia, dementia and attention deficit disorders. In one embodiment, the disorder is schizophrenia.

As used herein, the term “effective amount” means that amount of a drug or pharmaceutical agent that will elicit the biological or medical response of a tissue, system, animal or human that is being sought, for instance, by a researcher or clinician.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). The various subtypes of the disorders mentioned herein are contemplated as part of the present invention. Numbers in brackets after the listed diseases below refer to the classification code in DSM-IV.

In particular, the compounds of formula (I) may be of use in the treatment of schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-induced Psychotic Disorder including the subtypes With Delusions (293.81) and With Hallucinations (293.82); and Psychotic Disorder Not Otherwise Specified (298.9).

The compounds of formula (I) may be also of use in the treatment of mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-induced Mood Disorder (including the subtypes With Depressive Features, With Manic Features and With Mixed Features) and Mood Disorder Not Otherwise Specified (296.90).

The compounds of formula (I) may be of use in the treatment of anxiety disorders including Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-induced Anxiety Disorder and Anxiety Disorder Not Otherwise Specified (300.00).

The compounds of formula (I) may be of use in the treatment of substance-related disorders including Substance Use Disorders such as Substance Dependence and Substance Abuse; Substance-induced Disorders such as Substance Intoxication, Substance Withdrawal, Substance-induced Delirium, Substance-induced Persisting Dementia, Substance-induced Persisting Amnestic Disorder, Substance-induced Psychotic Disorder, Substance-induced Mood Disorder, Substance-induced Anxiety Disorder, Substance-induced Sexual Dysfunction, Substance-induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-induced Persisting Dementia, Alcohol-induced Persisting Amnestic Disorder, Alcohol-induced Psychotic Disorder, Alcohol-induced Mood Disorder, Alcohol-Induced Anxiety Disorder, Alcohol-induced Sexual Dysfunction, Alcohol-induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-induced Mood Disorder, Amphetamine-induced Anxiety Disorder, Amphetamine-induced Sexual Dysfunction, Amphetamine-induced Sleep Disorder and Amphetamine-Related Disorder Not Otherwise Specified (292.9); Caffeine Related Disorders such as Caffeine Intoxication (305.90), Caffeine-induced Anxiety Disorder, Caffeine-induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-induced Psychotic Disorder, Cannabis-induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-induced Psychotic Disorder, Cocaine-induced Mood Disorder, Cocaine-induced Anxiety Disorder, Cocaine-Induced Sexual Dysfunction, Cocaine-induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-induced Psychotic Disorder, Hallucinogen-induced Mood Disorder, Hallucinogen-induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-induced Persisting Dementia, Inhalant-induced Psychotic Disorder, Inhalant-induced Mood Disorder, Inhalant-induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of formula (I) may also be of use in the treatment of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-induced Sleep Disorder including the subtypes Insomnia Type, Hypersomnia Type, Parasomnia Type and Mixed Type.

The compounds of formula (I) may also be of use in the treatment of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of formula (I) may also be of use in the treatment of Autistic Disorder (299.00); Attention-Deficit /Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of formula (I) may also be of use in the treatment of Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9).

The compounds of formula (I) may also be of use in the treatment of cognitive impairment. Within the context of the present invention, the term cognitive impairment includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of the present invention may also be of use for the treatment of cognition impairment which arises in association or as a result of other diseases such as schizophrenia, bipolar disorder, depression, other psychiatric disorders and psychotic conditions associated with cognitive impairment.

The compounds of formula (I) may also be of use in the treatment of sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The compounds of formula (I) may also be of use as anticonvulsants. The compounds of formula (I) are thus useful in the treatment of convulsions in mammals, and particularly epilepsy in humans. “Epilepsy” is intended to include the following seizures: simple partial seizures, complex partial seizures, secondary generalised seizures, generalised seizures including absence seizures, myoclonic seizures, clonic seizures, tonic seizures, tonic clonic seizures and atonic seizures. The invention also provides a method of treating convulsions, which comprises administering to a mammal in need thereof an effective amount of a compound of formula (I) as hereinbefore described or a salt or solvate thereof. Treatment of epilepsy may be carried out by the administration of a non-toxic anticonvulsant effective amount of a compound of the formula (I) or a salt or solvate thereof.

The compounds of formula (I) may also be of use in the treatment of neuropathic pain, for example in diabetic neuropathy, sciatica, non-specific lower back pain, multiple sclerosis pain, fibromyalgia, HIV-related neuropathy, neuralgia such as post-herpetic neuralgia and trigeminal neuralgia and pain resulting from physical trauma, amputation, cancer, toxins or chronic inflammatory conditions.

The compounds of formula (I) and their salts and solvates thereof may also be suitable for combination with other active ingredients, such as typical and atypical antipsychotics, to provide improved treatment of psychotic disorders.

Thus, the present invention also provides:
i) a combination product comprising a compound of the invention and an antipsychotic;
ii) a pharmaceutical composition comprising a combination product as defined in i) above and at least one carrier, diluent or excipient;
iii) the use of a combination product as defined in i) above in the manufacture of a medicament for treating a disease or condition caused by a reduction or imbalance in glutamate receptor function in a mammal;
iv) a combination product as defined in i) above for use in treating a disease or condition caused by a reduction or imbalance in glutamate receptor function in a mammal;
v) a kit-of-parts for use in the treatment of a psychotic disorder comprising a first dosage form comprising a compound of the invention and one or more further dosage forms each comprising a antipsychotic agent for simultaneous therapeutic administration.
vi) a combination product as defined in i) above for use as a medicament;
vii) a method of treatment of a disease or condition caused by a reduction or imbalance in glutamate receptor function in a mammal comprising administering an effective amount of a combination product as defined in i) above.

The combination therapies of the invention may be administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a salt or solvate thereof and at least one antipsychotic agent are within the scope of the current invention. In one embodiment of adjunctive therapeutic administration as described herein, a patient is typically stabilised on a therapeutic administration of one or more of the of the components for a period of time and then receives administration of another component. Within the scope of this invention, the compounds of formula (I) or a salt or solvate thereof may be administered as adjunctive therapeutic treatment to patients who are receiving administration of at least one antipsychotic agent, but the scope of the invention also includes the adjunctive therapeutic administration of at least one antipsychotic agent to patients who are receiving administration of compounds of formula (I) or a salt or solvate thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a salt or solvate thereof to a patient receiving therapeutic administration of at least one antipsychotic agent. In a further aspect, the invention provides the use of compounds of formula (I) or a salt or solvate thereof in the manufacture of a medicament for adjunctive therapeutic administration for the treatment of a psychotic disorder in a patient receiving therapeutic administration of at least one antipsychotic agent. The invention further provides compounds of formula (I) or a salt or solvate thereof for use for adjunctive therapeutic administration for the treatment of a psychotic disorder in a patient receiving therapeutic administration of at least one antipsychotic agent.

In a further aspect, the invention provides a method of treatment of a psychotic disorder by adjunctive therapeutic administration of at least one antipsychotic agent to a patient receiving therapeutic administration of compounds of formula (I) or a salt or solvate thereof. In a further aspect, the invention provides the use of at least one antipsychotic agent in the manufacture of a medicament for adjunctive therapeutic administration for the treatment of a psychotic disorder in a patient receiving therapeutic administration of compounds of formula (I) or a salt or solvate thereof. The invention further provides at least one antipsychotic agent for adjunctive therapeutic administration for the treatment of a psychotic disorder in a patient receiving therapeutic administration of compounds of formula (I) or a salt or solvate thereof.

In a further aspect, the invention provides a method of treatment of a psychotic disorder by simultaneous therapeutic administration of compounds of formula (I) or a salt or solvate thereof in combination with at least one antipsychotic agent. The invention further provides the use of a combination of compounds of formula (I) or a salt or solvate thereof and at least one antipsychotic agent in the manufacture of a medicament for simultaneous therapeutic administration in the treatment of a psychotic disorder. The invention further provides the use of compounds of formula (I) or a salt thereof in the manufacture of a medicament for simultaneous therapeutic administration with at least one antipsychotic agent in the treatment of a psychotic disorder. The invention further provides compounds of formula (I) or a salt thereof for use for simultaneous therapeutic administration with at least one antipsychotic agent in the treatment of a psychotic disorder. The invention further provides the use of at least one antipsychotic agent in the manufacture of a medicament for simultaneous therapeutic administration with compounds of formula (I) or a salt thereof in the treatment of a psychotic disorder.

In further aspects, the invention provides a method of treatment of a psychotic disorder by simultaneous therapeutic administration of a pharmaceutical composition comprising compounds of formula (I) or a salt or solvate thereof and at least one mood stabilising or antimanic agent, a pharmaceutical composition comprising compounds of formula (I) or a salt or solvate thereof and at least one mood stabilising or antimanic agent, the use of a pharmaceutical composition comprising compounds of formula (I) or a salt or solvate thereof and at least one mood stabilising or antimanic agent in the manufacture of a medicament for the treatment of a psychotic disorder, and a pharmaceutical composition comprising compounds of formula (I) or a salt or solvate thereof and at least one mood stabilising or antimanic agent for use in the treatment of a psychotic disorder.

Examples of antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benzisothiazolyl-piperazines; triazine such as lamotrigine; dibenzoxazepines, such as loxapine; dihydroindolones, such as molindone; aripiprazole; and derivatives thereof that have antipsychotic activity.

Examples of tradenames and suppliers of selected antipsychotic drugs are as follows: clozapine (available under the tradename CLOZARIL®, from Mylan, Zenith Goldline, UDL, Novartis); olanzapine (available under the tradename ZYPREX®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from SmithKline Beecham (GSK); fluphenazine (available under the tradename PROLIXIN®, from Apothecon, Copley, Schering, Teva, and American Pharmaceutical Partners, Pasadena); thiothixene (available under the tradename NAVANE®, from Pfizer); trifluoperazine (10-[3-(4-methyl-1-piperazinyl)propyl]-2-(trifluoromethyl)phenothiazine dihydrochloride, available under the tradename STELAZINE®, from Smith Klein Beckman; perphenazine (available under the tradename TRILAFON®; from Schering); thioridazine (available under the tradename MELLARIL®; from Novartis, Roxane, HiTech, Teva, and Alpharma); molindone (available under the tradename MOBAN®, from Endo); and loxapine (available under the tradename LOXITANE®; from Watson). Furthermore, benperidol (Glianimon®), perazine (Taxilan®) or melperone (Eunerpan®)) may be used. Other antipsychotic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®;), prochlorperazine (available under the tradename COMPAZINE®), methotrimeprazine (available under the tradename NOZINAN®), pipotiazine (available under the tradename PIPOTRIL®), ziprasidone, and hoperidone.

It will be appreciated by those skilled in the art that the compounds according to the invention may advantageously be used in conjunction with one or more other therapeutic agents, for instance, antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

A pharmaceutical composition of the invention is usually adapted for oral, sub-lingual, buccal, parenteral (for example, subcutaneous, intramuscular, or intravenous), rectal, topical and intranasal administration and in forms suitable for administration by inhalation or insufflation (either through the mouth or nose). The most suitable means of administration for a particular patient will depend on the nature and severity of the conditions being treated and on the nature of the active compound. In one embodiment, oral administration is provided.

Formulations suitable for oral administration may be provided as discrete units, such as tablets, capsules, cachets, or lozenges, each containing a predetermined amount of the active compound; as powders or granules; as solutions or suspensions in aqueous or non-aqueous liquids; or as oil-in-water or water-in-oil emulsions.

Formulations suitable for sublingual or buccal administration include lozenges comprising the active compound and, typically, a flavoured base, such as sugar and acacia or tragacanth and pastilles comprising the active compound in an inert base, such as gelatin and glycerin or sucrose and acacia.

Formulations suitable for parenteral administration typically comprise sterile aqueous solutions containing a predetermined concentration of the active compound; the solution may be isotonic with the blood of the intended recipient. Such solutions may be administered intravenously or by subcutaneous or intramuscular injection.

Formulations suitable for rectal administration may be provided as unit-dose suppositories comprising the active ingredient and one or more solid carriers forming the suppository base, for example, cocoa butter.

Formulations suitable for topical or intranasal application include ointments, creams, lotions, pastes, gels, sprays, aerosols and oils. Suitable carriers for such formulations include petroleum jelly, lanolin, polyethylene glycols, alcohols, and combinations thereof.

The formulations of the invention may be prepared by any suitable method, typically by uniformly and intimately admixing the active compound(s) with liquids or finely divided solid carriers, or both, in the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Aqueous solutions for parenteral administration are typically prepared by dissolving the active compound in sufficient water to give the desired concentration and then rendering the resulting solution sterile and isotonic.

It will be appreciated that the precise dose administered will depend on the age and condition of the patient and the frequency and route of administration and will be at the ultimate discretion of the attendant physician. The compound may be administered in single or divided doses and may be administered one or more times, for example 1 to 4 times per day.

The invention is further illustrated by the following non-limiting examples.

### ABBREVIATIONS

DCM dichloromethane

DMF dimethylformamide

iPrOH isopropyl alcohol

### Analytical LC/MS Chromatography Conditions for Examples 1-26

| | |
|---|---|
| Column: | Waters Atlantis 50 mm × 4.6 mm, 3 um particle |
| | size |
| Mobile phase: | A: 0.05% Formic acid + Water |
| | B: Acetonitrile +0.05% Formic acid |
| Gradient: | 5-min runtime: 3% B to 97% B over 4 min |
| Flow rate: | 3 ml/min |
| UV wavelength range: | 220-330 nm |
| Temperature: | 30° C. |

### Analytical LC/MS Chromatography Conditions for Examples 27-32

| | |
|---|---|
| Column: | Waters Acquity 50 mm × 2.1 mm, 1.7 um particle |
| | size |
| Mobile phase: | A: Water + 0.05% Formic acid |
| | B: Acetonitrile + 0.05% Formic acid |
| Gradient: | 2 -min runtime: 3% B to 97% B over 1.3 min |
| Flow rate: | 1 ml/min |
| UV wavelength range: | 220-330 nm |
| Temperature: | 40° C. |

### Mass Directed Auto-Purification System (MDAP) Conditions

| | |
|---|---|
| Column: | Waters Atlantis 19 mm × 100 mm or 30 mm × 100 mm, |
| | 5 um particle size |
| Mobile phase: | A: 0.1% Formic acid + Water |
| | B: Acetonitrile +0.1% Formic acid |
| Gradient: | 13.5 min runtime with 10 min gradient dependant on |
| | analytical retention time |
| Flow rate: | 20 or 40 ml/min |

MDAP refers to purification by HPLC, wherein fraction collection is triggered by detection of the programmed mass ion for the compound of interest.

Where reactions are described as having been carried out in a similar manner to earlier, more completely described reactions, the general reaction conditions used were essentially the same. Work up conditions used were of the types standard in the art, but may have been adapted from one reaction to another. In the procedures that follow, reference to the product of a Description or Example by number is typically provided. This is provided merely for assistance to the skilled chemist to identify the starting material used. The starting material may not necessarily have been prepared from the batch referred to. All reactions were either carried out under argon or may be carried out under argon, unless otherwise stated.

### Description 1: Methyl amino(4-chlorophenyl)acetate

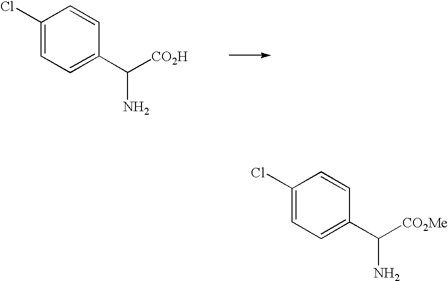

To ice-chilled methanol (300 ml) under argon was carefully added dropwise thionyl chloride (15.44 ml; 0.217 mol) over 45 min. 4-Chlorophenylglycine (26.26 g; 0.141 mol) was added, ice cooling removed and the reaction mixture warmed to 40° C.; the reaction was stirred at 40° C. for 48 h. After cooling to room temperature, the reaction was evaporated under reduced pressure. Re-evaporation from methanol afforded a white solid which was triturated with diethyl ether (ca. 700 ml) and then stored at ca. 4° C. for 64 h, filtered, washed with diethyl ether and dried in vacuo to afford the title product as the hydrochloride salt (33.40 g; 100%). ¹H NMR (d₆-DMSO) δ: 3.72 (3H, s), 5.36 (1H, s), 7.53-7.58 (4H, m), 9.07 (3H, s). Mass Spectrum (Electrospray LC/MS): Found 200 (MH⁺). C₉H₁₀³⁵ClNO₂ requires 199. Ret. time 1.32 min.

### Description 2: 2-Amino-2-(4-chlorophenyl)acetamide

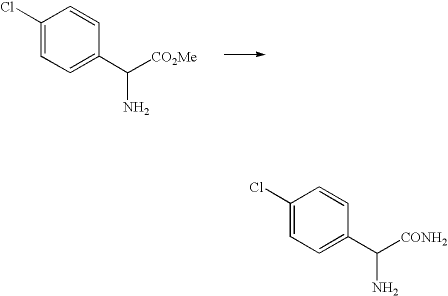

Methyl amino(4-chlorophenyl)acetate D1 as the hydrochloride salt (33.40 g; 0.14 mol) was dissolved in 0.88 ammonia (500 ml; ca. 7.4 mol) and stirred at room temperature for 64 h. The reaction mixture was extracted with DCM (300 ml×6), the extracts dried (Na₂SO₄) and evaporated under reduced pressure to a white solid, which was dried under reduced pressure to afford the title product (22.45 g; 86%). ¹H NMR (CDCl₃) δ: 1.82 (2H, br s), 4.53 (1H, s), 5.49 (1H, br s), 6.92 (1H, br s), 7.32-7.39 (4H, m).

### Description 3: 3-(4-Chlorophenyl)-1,4-diazaspiro[4.5]decan-2-one

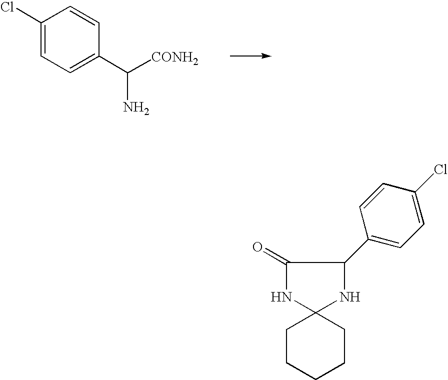

To 2-amino-2-(4-chlorophenyl)acetamide D2 (10.00 g; 54.3 mmol) in methanol (500 ml) was added cyclohexanone (5.62 ml; 54.3 mmol) and H—Y zeolites (10.00 g) and the mixture stirred under reflux for 24 h. The reaction was allowed to cool to room temperature and after 4 days was filtered and the solid washed well with methanol. The filtrate was evaporated to afford the title product (12.91 g; 90%) as a white solid. ¹H NMR (CDCl₃) δ: 1.44-1.57 (4H, m), 1.66-1.76 (6H, m), 2.21 (1H, s), 4.69 (1H, s), 6.80 (1H, s), 7.32-7.35 (2H, m), 7.45-7.49 (2H, m).

### Description 4: 3-(4-Chlorophenyl)-1,4-diazaspiro[4.5]dec-3-en-2-one

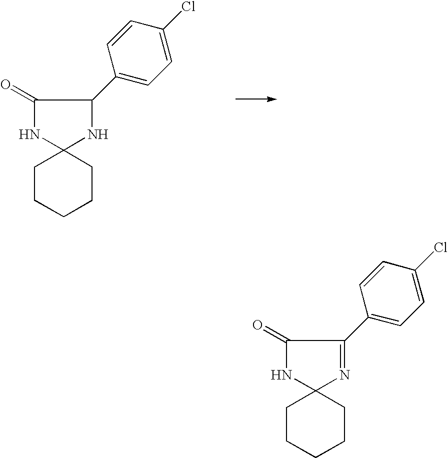

N-Bromosuccinimide (8.69 g; 48.81 mmol) was added in one portion to a stirred solution of 3-(4-chlorophenyl)-1,4-diazaspiro[4.5]decan-2-one D3 (12.91 g; 48.81 mmol) in DCM (400 ml) and the mixture stirred overnight at room temperature. Saturated aqueous sodium bicarbonate (500 ml) was added and the mixture stirred for 0.5 h. The layers were separated and the aqueous extracted with DCM (300 ml). The combined organics were dried (Na₂SO₄) and the solvent removed under reduced pressure at 45° C. The residual solid was partitioned between DCM (500 ml) and saturated aqueous sodium bicarbonate (500 ml) and stirred overnight at room temperature. The aqueous layer was extracted with DCM (300 ml) and the organic layers combined, dried (Na₂SO₄) and the solvent removed under reduced pressure to afford the title product (10.25 g; 80%) as a pale yellow solid. ¹H NMR (CDCl₃) δ: 1.51-1.70 (6H, m), 1.91-1.99 (4H, m), 7.42-7.49 (2H, m), 8.36-8.39 (2H, m), 8.88 (1H, s).

### Description 5: Ethyl[3-(4-chlorophenyl)-2-oxo-1,4-diazaspiro[4.5]dec-3-en-1-yl]acetate

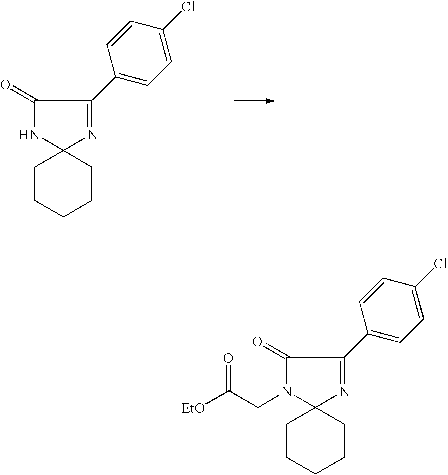

A mixture of 3-(4-chlorophenyl)-1,4-diazaspiro[4.5]dec-3-en-2-one D4 (4.06 g; 15.5 mmol ethyl bromoacetate (8.55 ml; 77.3 mmol) and potassium carbonate (2.35 g; 17.0 mmol) in acetone (200 ml) was heated and stirred for a total of 2 days and one night. 2.35 g of potassium carbonate and 3 ml of ethyl bromoacetate were added. After one night under stirrer and heating the reaction mixture was cooled and partitioned between water and DCM. The DCM layer was separated, evaporated and the residue chromatographed twice, eluting with ethyl acetate and ethyl acetate-pentane mixtures to afford the title product (3.2 g; 59%). ¹H NMR (CDCl₃) δ:1.24-1.34 (5H, m), 1.43-1.47 (2H, m), 1.74-1.82 (4H, m), 1.88-1.91 (1H, m), 1.97-2.07 (1H, m), 4.16 (2H, s), 4.20-4.27 (2H, m), 7.41-7.45 (2H, m), 8.43-8.46 (2H, m).

### Description 6: [3-(4-Chlorophenyl)-2-oxo-1,4-diazaspiro[4.5]dec-3-en-1-yl]acetic acid

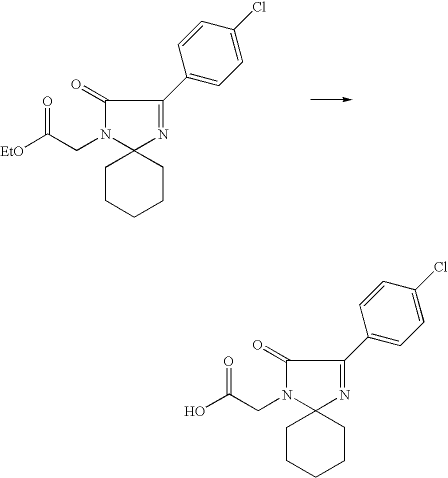

To a stirred mixture of ethyl[3-(4-chlorophenyl)-2-oxo-1,4-diazaspiro[4.5]dec-3-en-1-yl]acetate D5 (4.240 g; 12.17 mmol) in water (150 ml) and methanol (50 ml) was added sodium hydroxide (0.584 g; 14.6 mmol) and the reaction heated at 60° C. overnight, cooled and evaporated under reduced pressure. The residue was partitioned between water (400 ml) and ethyl acetate (200 ml). The aqueous layer was acidified with 5N HCl and extracted into DCM (150 ml×3). The combined DCM extracts were dried (Na₂SO₄) and evaporated under reduced pressure to afford the title acid (3.42 g; 88%) as a colourless solid. ¹H NMR (CDCl₃) δ: 1.25-1.35 (1H, m), 1.44-1.47 (2H, m), 1.76-2.07 (7H, m), 4.22 (2H, s), 7.10-7.70 (1H, br s), 7.41-7.44 (2H, m), 8.39-8.43 (2H, m).

### Description 7: [3-(4-Chlorophenyl)-2-oxo-1,4-diazaspiro[4.5]dec-3-en-1-yl]acetyl chloride

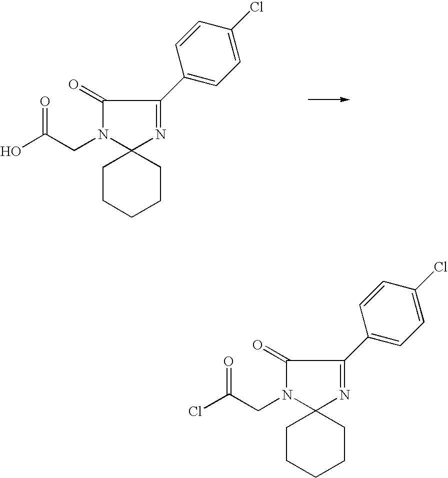

To a suspension of [3-(4-chlorophenyl)-2-oxo-1,4-diazaspiro[4.5]dec-3-en-1-yl]acetic acid D6 (700 mg; 2.18 mmol) in DCM (40 ml) was added oxalyl chloride (0.4 ml; 608 mg; 4.80 mmol) followed with stirring by DMF (1 drop). After stirring overnight the reaction was evaporated under reduced pressure to afford the title product (700 mg) as a pale yellow solid which was used without further purification. Mass Spectrum (Electrospray LC/MS; MeOH): Found 335 (MH⁺ for methyl ester). C₁₇H₁₉³⁵ClN₂O₃ requires 334. Ret. time 3.5 min.

### Description 8: 2-Amino-2-[4-(methyloxy)phenyl]acetamide

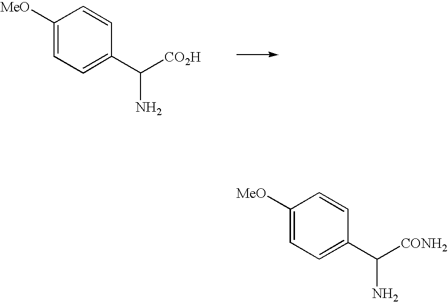

To an ice-cold suspension of 4-methoxyphenylglycine (3.77 g; 0.021 mol) in methanol was added thionyl chloride dropwise over 30 min. After complete addition, the reaction mixture was heated at reflux for 3 h, cooled and evaporated. The resulting solid was dissolved in 0.88 ammonia (100 ml) and stirred at room temperature overnight. The reaction was extracted twice with DCM and the organic phases separated with a Phase-Separation cartridge and evaporated under reduced pressure to afford the title product (0.45 g; 12%) as a white solid. ¹H NMR (CDCl₃) δ: 1.77 (2H, br s), 3.80 (3H, s), 4.50 (1H, s), 5.52 (1H, s), 6.83 (1H, s), 6.87-6.91 (2H, m), 7.33-7.36 (2H, m).

### Description 9: 3-[4-(Methyloxy)phenyl]-1,4-diazaspiro[4.5]decan-2-one

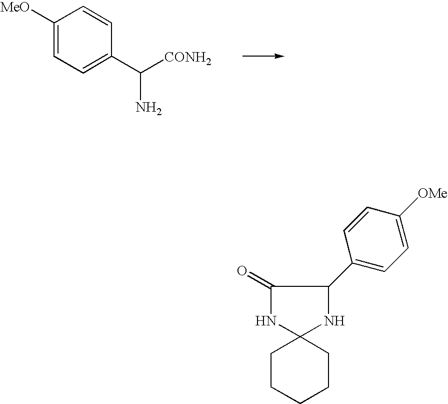

The title compound (0.420 g; 65%) was obtained from 2-amino-2-[4-(methyloxy)phenyl]acetamide D8 (0.450 g; 2.5 mmol), cyclohexanone (0.245 g; 2.5 mmol) and H—Y zeolites (1 g) in methanol (20 ml) in a similar manner to that described in D3. ¹H NMR (CDCl₃) δ: 1.44-1.57 (4H, m), 1.71-1.73 (6H, m), 2.11 (1H, br s), 3.80 (3H, s), 4.64 (1H, s), 6.55 (1H, br s), 6.89-6.92 (2H, m), 7.36-7.40 (2H, m).

### Description 10: 3-[4-(Methyloxy)phenyl]-1,4-diazaspiro[4.5]dec-3-en-2-one

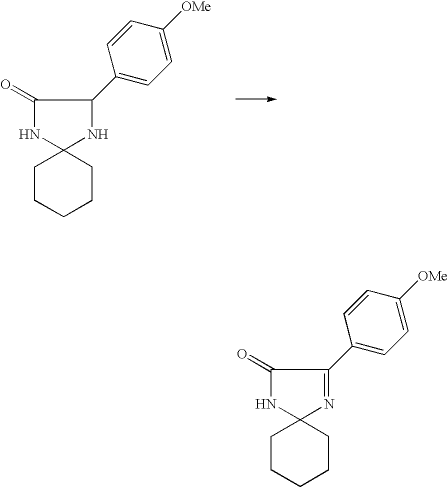

The title product (406 mg; 100%) was obtained from 3-[4-(methyloxy)phenyl]-1,4-diazaspiro[4.5]decan-2-one D9 (400 mg; 1.54 mmol) and N-bromosuccinimide (275 mg; 1.55 mmol) in DCM (20 ml) using a method similar to that described in D4. ¹H NMR (CDCl₃) δ: 1.40-1.75 (6H, m), 1.85-2.00 (4H, m), 3.87 (3H, s), 6.94-6.98 (2H, m), 8.18 (1H, brs), 8.37-8.40 (2H, m).

### Description 11: Ethyl{3-[4-(methyloxy)phenyl]-2-oxo-1,4-diazaspiro[4.5]dec-3-en-1-yl}acetate

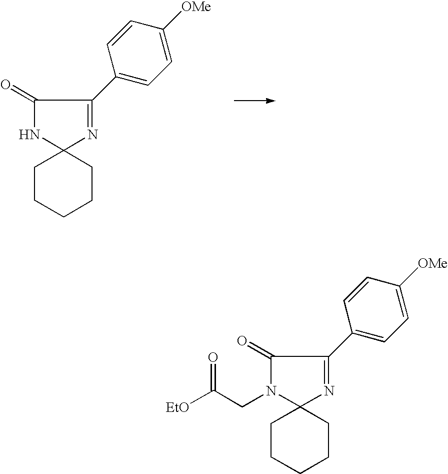

A mixture of 3-(4-methoxyphenyl)-1,4-diazaspiro[4.5]dec-3-en-2-one D10 (400 mg; 1.55 mmol), ethyl bromoacetate (0.7 ml; 6.30 mmol) and potassium carbonate (260 mg; 1.86 mmol) in acetone (200 ml) was heated at reflux for ca. 64 h; further ethyl bromoacetate (0.7 ml; 6.30 mmol) was added and heating continued for 48 h, after which further potassium carbonate (260 mg; 1.86 mmol) and ethyl bromoacetate (0.7 ml; 6.30 mmol) was added and heating continued for a further 48 h. Acetone was evaporated and the residue partitioned between DCM and water. The organic phase was separated and dried using a Phase-separation cartridge and evaporated, and the residue chromatographed eluting with ethyl acetate-pentane gradient to afford the title product (326 mg; 61%). ¹H NMR (CDCl₃) δ: 1.23-1.33 (4H, m), 1.42-1.45 (2H, m), 1.72-1.80 (4H, m), 1.87-1.90 (1H, s), 1.98-2.09 (2H, m), 3.87 (3H, s), 4.16 (2H, s), 4.19-4.25 (2H, s), 6.94-7.26 (2H, m), 8.45-8.48 (2H, m).

### Description 12: {3-[4-(Methyloxy)phenyl]-2-oxo-1,4-diazaspiro[4.5]dec-3-en-1-yl}acetic acid

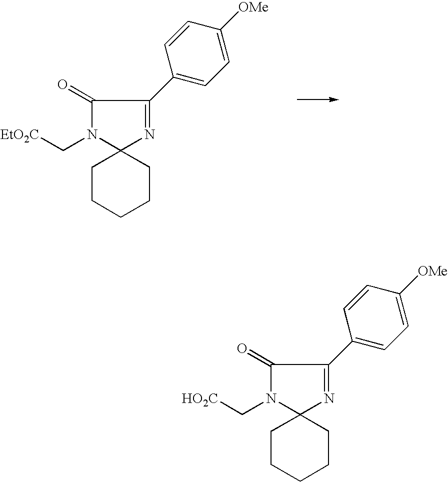

To ethyl{3-[4-(methyloxy)phenyl]-2-oxo-1,4-diazaspiro[4.5]dec-3-en-1-yl}acetate D11 (326 mg; 0.95 mmol) in ethanol (16 ml) and water (4 ml) was added sodium hydroxide (80 mg; 2.00 mmol) and the mixture heated at reflux for 16 h. The reaction was cooled and evaporated to give a slurry that was acidified with 2N HCl and extracted with ethyl acetate (×2). The organics were separated and dried with a little Na₂SO₄ and passed through a Phase-separation cartridge to afford the title product (290 mg; 97%). ¹H NMR (CDCl₃) δ:

1.20-1.33 (1H, m), 1.43-1.46 (2H, m), 1.74-1.80 (4H, m), 1.87-1.90 (1H, m), 1.98-2.18 (2H, m), 3.86 (3H, s), 4.22 (2H, s), 6.94-6.96 (2H, m), 8.42-8.44 (2H, m), 9.75 (1H, brs).

### Description 13: {3-[4-(Methyloxy)phenyl]-2-oxo-1,4-diazaspiro[4.5]dec-3-en-1-yl}acetyl chloride

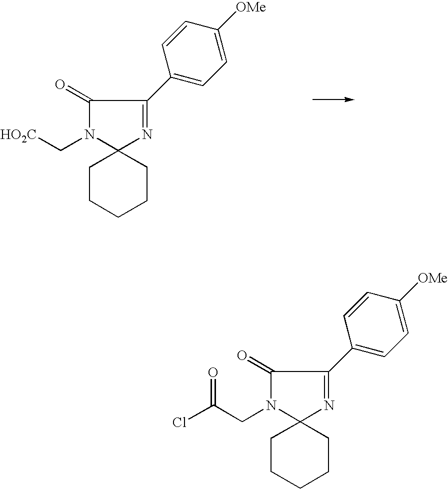

A mixture of {3-[4-(methyloxy)phenyl]-2-oxo-1,4-diazaspiro[4.5]dec-3-en-1-yl}acetic acid D12 (288 mg; 0.9 mmol), oxalyl chloride (0.18 ml; 2.1 mmol) and DMF (1 drop) in DCM (20 ml) was stirred at room temperature for 2 h. The reaction was evaporated and triturated with toluene (×2) to afford the title product as an orange solid which was used without further purification.

### Description 14: 2-Bromo-N-(3,5-difluorophenyl)acetamide

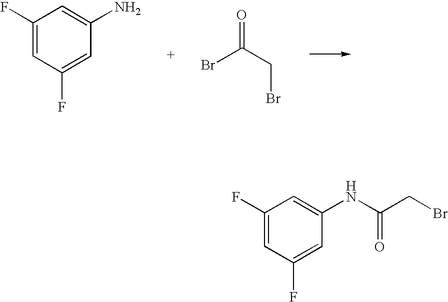

A mixture of 3,5-difluoroaniline (10 g; 77.45 mmol) and bromoacetyl bromide (6.73 ml; 77.45 mmol) in anhydrous dioxan (100 ml) was refluxed for 1.5 h, cooled to room temperature and diluted with water (400 ml) to afford a gum. The mother liquors were decanted and water added, followed by ethyl acetate. After stirring for 10 min the layers were separated and the organics dried and evaporated under reduced pressure. Recrystallisation from ethyl acetate-pentane afforded the title product as pale yellow crystals (6.5 g; 33%). ¹H NMR (CDCl₃) δ: 4.02 (2H, s), 6.60-6.65 (1H, m), 7.14-7.20 (2H, m), and 8.16 (1H, br s).

### Description 15: 3-[4-(Methyloxy)phenyl]-1,4-diazaspiro[4.4]nonan-2-one

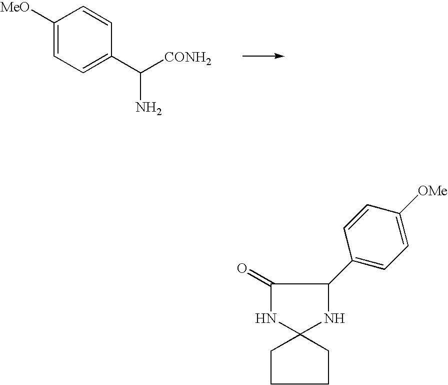

The title compound (1.92 g; 56%) was obtained from 2-amino-2-[4-(methyloxy)phenyl]acetamide D8 (2.50 g; 1.39 mmol), cyclopentanone (1.24 ml; 1.39 mmol) and H—Y zeolites (5 g) in ethanol (200 ml) in a similar manner to that described in D3. Mass Spectrum (Electrospray LC/MS): Found 247 (MH⁺). C₁₄H₁₈N₂O₂ requires 246. Ret. time 1.28/1.33 min.

### Description 16: 3-[4-(Methyloxy)phenyl]-1,4-diazaspiro[4.4]non-3-en-2-one

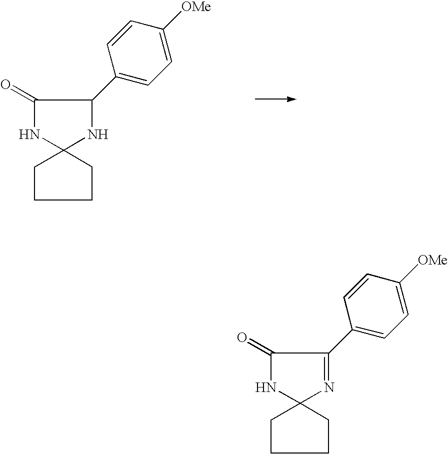

The title product (1.92 g; 100%) was obtained from 3-[4-(methyloxy)phenyl]-1,4-diazaspiro[4.4]nonan-2-one D15 (1.92 g; 7.8 mmol) and N-bromosuccinimide (1.40 g; 7.8 mmol) in DCM (150 ml) using a method similar to that described in D4. ¹H NMR (CDCl₃) inter alia δ: 1.80-2.20 (8H, m), 3.87 (3H, s), 6.94-6.97 (2H, m), 7.98 (1H, br s), 8.36-8.40 (2H, m). Mass Spectrum (Electrospray LC/MS): Found 245 (MH⁺). C₁₄H₁₆N₂O₂ requires 244. Ret. time 2.45 min.

### Description 17: 3-(4-chlorophenyl)-1,4-diazaspiro[4.4]nonan-2-one

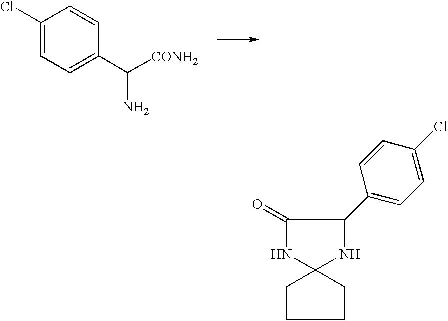

The title compound (combined yield from 2 crops 1.635 g; 56%) was obtained from 2-amino-2-[4-(chloro)phenyl]acetamide D2 (2.40 g; 13 mmol), cyclopentanone (1.15 ml; 13 mmol) and H—Y zeolites (3.13 g) in ethanol (140 ml) in a similar manner to that described in D3. After removal of solvent, final compound was obtained by trituration with ethanol to give title compound 1.45 g). A second crop (0.185 g) was obtained from the ethanol mother liquors. ¹H NMR (CDCl₃) inter alia δ: 1.66-2.00 (8H, m), 2.30 (1H, m), 4.64 (1H, m), 6.54 (1H, s), 7.35 (2H, d) 7.45 (2H, d)

### Description 18: 3-(4-chlorophenyl)-1,4-diazaspiro[4.4]non-3-en-2-one

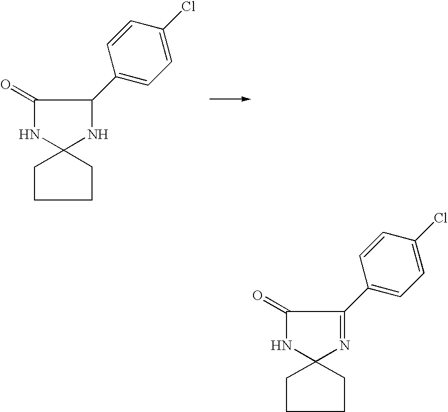

The title product (1.05; 72%) after recrystallisation from ethanol, was obtained from 3-(4-chlorophenyl)-1,4-diazaspiro[4.4]nonan-2-one D18 (1.48 g; 5.90 mmol) and N-bromosuccinimide (1.05 g; 5.90 mmol) in DCM (50 ml) using a method similar to that described in D4. ¹H NMR (CDCl₃) inter alia δ: 1.86-2.14 (8H, m), 7.43 (2H, d) and 8.37 (2H, m).

The following bromo(substituted aryl)acetamides are either known in the literature or were prepared according to the method of Description 14:

| Desc. | Structure | NMR | Name |
|---|---|---|---|
| 19 | 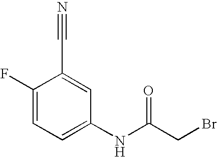 | ¹H NMR δ (CDCl₃, 400 MHz), 4.05 (2 H, s), 7.21 (1 H, m), 7.73 (1 H, m), 7.95 (1 H, q), 8.19 (1 H, s) | 2-bromo-N-(3-cyano- 4- fluorophenyl) acetamide |
| 20 | 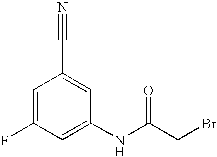 | ¹H NMR δ (CDCl₃, 400 MHz), 4.06 (2 H, s), 7.63 (1 H, s), 7.74 (1 H, dd) 8.26 (1 H, s) | 2-bromo-N-(3-cyano- 5- fluorophenyl) acetamide |
| 21 | 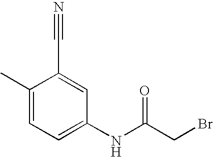 | ¹H NMR δ (CDCl₃, 400 MHz), 2.53 (2 H, s), 7.31 (1 H, d), 7.61 (1 H, dd), 7.89 *1 H, d), 8.15 (1 H, s) | 2-bromo-N-(3-cyano- 4- methylphenyl) acetamide |

### EXAMPLE 1

### 2-[3-(4-Chlorophenyl)-2-oxo-1,4-diazaspiro[4.5]dec-3-en-1-yl]-N-(3,5-difluorophenyl)acetamide

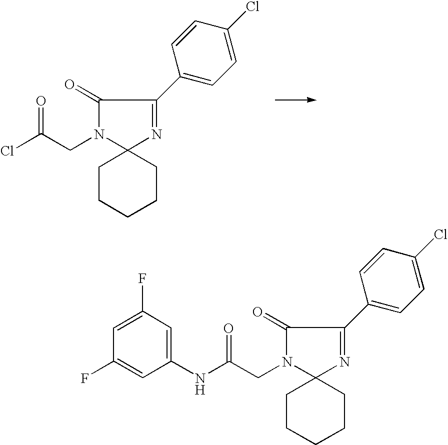

A mixture of 3,5-difluoroaniline (42 mg; 0.33 mmol), [3-(4-chlorophenyl)-2-oxo-1,4-diazaspiro[4.5]dec-3-en-1-yl]acetyl chloride D7 (105 mg; 0.31 mmol), and triethylamine (0.1 ml; 0.72 mmol) in DCM (2 ml) was shaken for 16 h. Saturated aqueous sodium hydrogencarbonate was added and the mixture shaken for 5 min and then separated and dried using a Phase-separation cartridge. The organics were evaporated and purified using mass directed auto-purification chromatography to afford the title product (37 mg; 28%). ¹H NMR (CDCl₃) δ: 1.29-1.42 (3H, m), 1.82-2.08 (7H, m), 4.23 (2H, s), 6.51-6.57 (1H, m), 7.08-7.13 (2H, m), 7.45-7.49 (2H, m), 8.41-8.44 (2H, m), 9.14 (1H, s). Mass Spectrum (Electrospray LC/MS): Found 432 (MH⁺). C₂₂H₂₀³⁵ClF₂N₃O₂ requires 431. Ret. time 3.78 min.

### EXAMPLE 2

### 2-{3-[4-(Methyloxy)phenyl]-2-oxo-1,4-diazaspiro[4.5]dec-3-en-1-yl}-N-[3-(trifluoromethyl)phenyl]acetamide

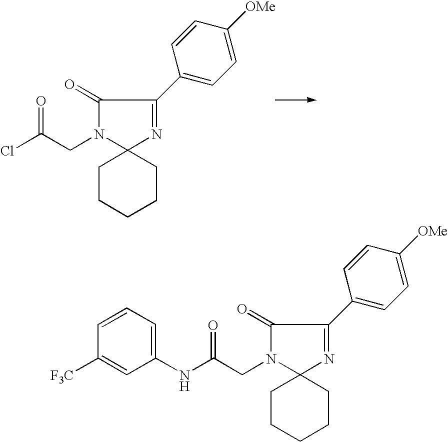

The title compound (27 mg; 33%) was obtained from {3-[4-(methyloxy)phenyl]-2-oxo-1,4-diazaspiro[4.5]dec-3-en-1-yl}acetyl chloride D13 (60 mg; 0.18 mmol), 3-trifluoromethylaniline (32 mg; 0.2 mmol) and triethylamine (0.1 ml; 0.72 mmol) in DCM (4 ml) using a method similar to that described in E1. ¹H NMR (CDCl₃) δ: 1.34-1.37 (3H, m), 1.83-2.05 (7H, m), 3.89 (3H, s), 4.25 (2H, s), 6.98-7.02 (2H, m), 7.34-7.35 (1H, m), 7.39-7.43 (1H, m), 7.65-7.67 (1H, m), 7.81 (1H, m), 8.43-8.47 (2H, m), 9.16 (1H, s). Mass Spectrum (Electrospray LC/MS): Found 460 (MH⁺). C₂₄H₂₄F₃N₃O₃ requires 459. Ret. time 3.61 min.

### EXAMPLE 3

### 2-[3-(4-Chlorophenyl)-2-oxo-1,4-diazaspiro[4.5]dec-3-en-1-yl]-N-(2-cyanophenyl)acetamide

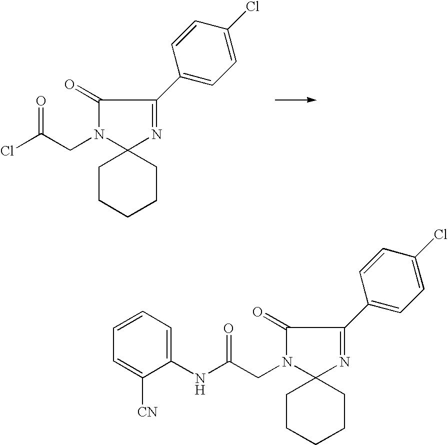

The title compound (77 mg; 59%) was obtained from [3-(4-chlorophenyl)-2-oxo-1,4-diazaspiro[4.5]dec-3-en-1-yl]acetyl chloride D7 (106 mg; 0.31 mmol), 2-cyanoaniline (57 mg; 0.48 mmol) and triethylamine (0.22 ml; 1.55 mmol) in DCM (4 ml) using a method similar to that described for E1, except that the product was isolated using silica gel chromatography, eluting with an ethyl acetate-pentane gradient. Mass Spectrum (Electrospray LC/MS): Found 421 (MH⁺). C₂₃H₂₁³⁵ClN₄O₂ requires 420. Ret. time 3.50 min.

Anilines, benzoic acids, benzoyl chlorides and arylglycine starting materials were available commercially, unless otherwise stated.

The compounds in table 1 below were prepared using similar methods to those described for the Examples above. Method: A=Acid chloride (using method similar to that in Example 1). Work-up and purification was carried out using appropriate methods similar to those described in the examples above.

TABLE 1

| | | | Mass spectrum | |
|---|---|---|---|---|
| | | | (Electrospray | |
| | | | LC/MS), API* | |
| Ex | Structure | Method | Ret. time (min) | Name |
| 4 | 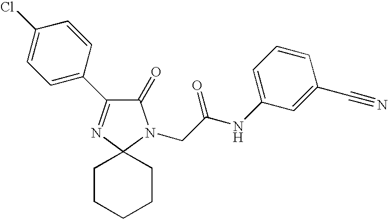 | A | Found 421 (MH⁺) C₂₃H₂₁³⁵ClN₄O₂ requires 420; 3.45. | 2-[3-(4-chlorophenyl)-2- oxo-1,4- diazaspiro[4.5]dec-3-en- 1-yl]-N-(3- cyanophenyl)acetamide |
| 5 | 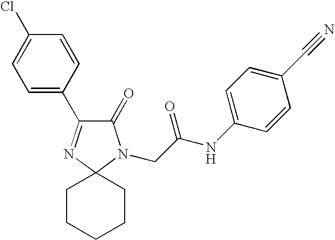 | A | Found 421 (MH⁺) C₂₃H₂₁³⁵ClN₄O₂ requires 420; 3.45. | 2-[3-(4-chlorophenyl)-2- oxo-1,4- diazaspiro[4.5]dec-3-en- 1-yl]-N-(4- cyanophenyl)acetamide |
| 6 | 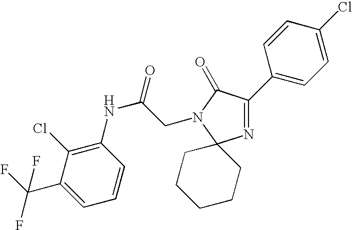 | A | Found 498 (MH⁺) C₂₃H₂₀³⁵Cl₂F₃N₃O₂ requires 497; 3.91. | 2-[3-(4-chlorophenyl)-2- oxo-1,4-diazaspiro[4.5] dec-3-en-1-yl]-N-[2- chloro-3-(trifluoromethyl) phenyl]acetamide |
| 7 | 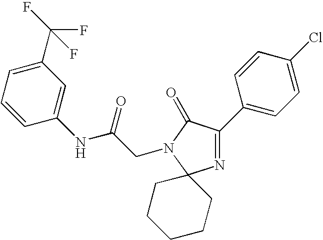 | A | Found 464 (MH⁺) C₂₃H₂₁³⁵ClF₃N₃O₂ requires 463; 3.76. | 2-[3-(4-chlorophenyl)-2- oxo-1,4-diazaspiro[4.5] dec-3-en- 1-yl]-N-[3-(trifluoromethyl) phenyl]acetamide |
| 8 | 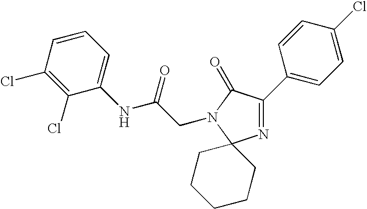 | A | Found 464 (MH⁺) C₂₂H₂₀³⁵Cl₃N₃O₂ requires 463; 3.96. | 2-[3-(4-chlorophenyl)-2- oxo-1,4- diazaspiro[4.5]dec-3-en- 1-yl]-N-(2,3- dichlorophenyl)acetamide |
| 9 | 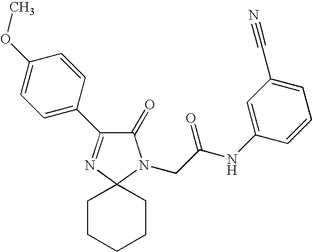 | A | Found 417 (MH⁺) C₂₄H₂₄N₄O₃ requires 416; 3.27. | N-(3-cyanophenyl)-2-{3- [4-(methyloxy)phenyl]-2- oxo-1,4- diazaspiro[4.5]dec-3-en- 1-yl}acetamide |
| 10 | 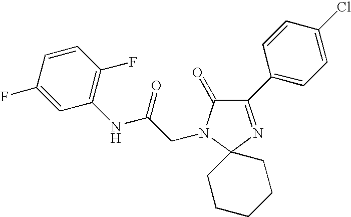 | A | Found 432 (MH⁺) C₂₂H₂₀³⁵ClF₂N₃O₂ requires 431; 3.78. | 2-[3-(4-chlorophenyl)-2- oxo-1,4- diazaspiro[4.5]dec-3-en- 1-yl]-N-(2,5- difluorophenyl)acetamide |

### EXAMPLE 11

### N-(3,5-Difluorophenyl)-2-{3-[4-(methyloxy)phenyl]-2-oxo-1,4-diazaspiro[4.5]dec-3-en-1-yl}acetamide

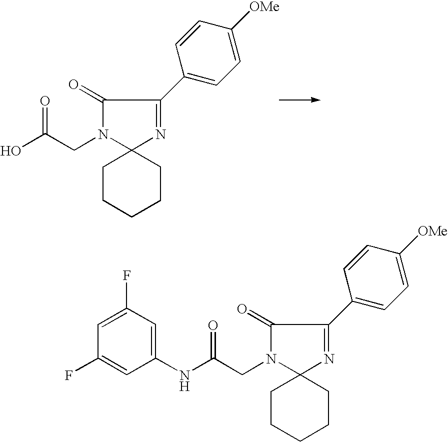

{3-[4-(Methyloxy)phenyl]-2-oxo-1,4-diazaspiro[4.5]dec-3-en-1-yl}acetyl chloride D13, prepared from {3-[4-(methyloxy)phenyl]-2-oxo-1,4-diazaspiro[4.5]dec-3-en-1-yl}acetic acid D12 (100 mg; 0.316 mmol), oxalyl chloride (33 ul; 0.38 mmol) and DMF (1 drop) in DCM (5 ml) in a similar manner to that described in D13, was reacted with 3,5-difluoroaniline (41 mg; 0.316 mmol) and triethylamine (88 ul; 0.63 mmol) in DCM (3 ml) at room temperature for 3 days. The reaction mixture was loaded onto silica gel and eluted with a 0-50% ethyl acetate-pentane gradient to afford the title product (95 mg; 70%). ¹H NMR (CDCl₃) δ: 1.33-1.35 (3H, m), 1.81-2.05 (7H, m), 3.89 (3H, s), 4.22 (2H, s), 6.51-6.56 (1H, m), 6.98-7.01 (2H, m), 7.10-7.14 (2H, m), 8.43-8.46 (2H, m), 9.23 (1H, s). Mass Spectrum (Electrospray LC/MS): Found 428 (MH⁺). C₂₃H₂₃F₂N₃O₃ requires 427. Ret. time 3.53 min.

### EXAMPLE 12

### N-(3,5-Difluorophenyl)-2-{3-[4-(methyloxy)phenyl]-2-oxo-1,4-diazaspiro[4.4]non-3-en-1-yl}acetamide

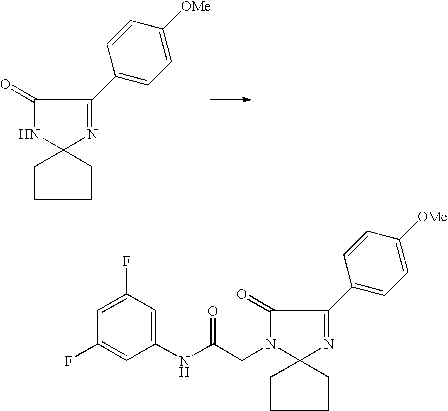

Sodium hydride (20 mg; 60% dispersion in oil; 0.499 mmol) was added in one portion to a stirred solution of 3-[4-(methyloxy)phenyl]-1,4-diazaspiro[4.4]non-3-en-2-one D16 (103 mg; 0.422 mmol) in DMF (3 ml) under argon. The mixture was stirred at room temperature for 5 min prior to the addition of 2-bromo-N-(3,5-difluorophenyl) acetamide D14 (88 mg; 0.353 mmol) in one portion. The reaction was stirred at room temperature for 3 h and then saturated aqueous sodium bicarbonate solution (50 ml) was added. After 18 h, the mixture was diluted with saturated aqueous sodium bicarbonate solution (200 ml) and extracted with ethyl acetate (2×150 ml). The combined organics were dried over sodium sulphate and evaporated under reduced pressure. The residue was purified in 2 batches using mass directed auto-purification chromatography to afford the title product (85 mg; 49%). ¹H NMR (CDCl₃) δ: 1.81-1.86 (2H, m), 1.97-2.16 (6H, m), 3.88 (3H, s), 4.23 (2H, s), 6.51-6.57 (1H, m), 6.97-7.01 (2H, m), 7.09-7.15 (2H, m), 8.38-8.42 (2H, m), 9.25 (1H, s). Mass Spectrum (Electrospray LC/MS): Found 414 (MH⁺). C₂₂H₂₁F₂N₃O₃ requires 413. Ret. time 3.26 min.

The compounds in table 2 below were prepared using similar methods to those described for the examples above. Method: A=Acid chloride (using method similar to that in Example 1). Method B=Alkylation (using a method similar to that for Example 12). Work-up and purification was carried out using appropriate methods similar to those described in the examples above.

TABLE 2

| | | | Mass spectrum | |
|---|---|---|---|---|
| | | | (Electrospray | |
| | | | LC/MS), API* | |
| Ex | Structure | Method | Ret. time (min) | Name |
| 13 | 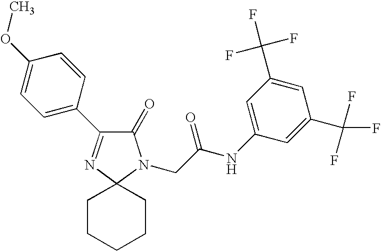 | A | Found 528 (MH⁺) C₂₅H₂₃F₆N₃O₃ requires 527; 3.86. | N-[3,5- bis(trifluoromethyl)phenyl]-2- {3-[4-(methyloxy)phenyl]-2- oxo-1,4-diazaspiro[4.5]dec-3- en-1-yl}acetamide |
| 14 | 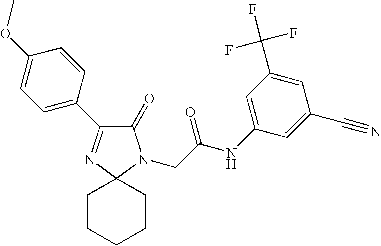 | A | Found 485 (MH⁺) C₂₅H₂₃F₃N₄O₃ requires 484; 3.59. | N-[3-cyano-5- (trifluoromethyl)phenyl]-2-{3- [4-(methyloxy)phenyl]-2-oxo- 1,4-diazaspiro[4.5]dec-3- en-1-yl}acetamide |
| 15 | 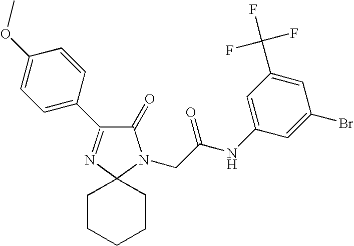 | A | Found 538 (MH⁺) C₂₄H₂₃BrF₃N₃O₃ requires 537; 3.86. | N-[3-bromo-5- (trifluoromethyl)phenyl]-2-{3- [4-(methyloxy)phenyl]-2-oxo- 1,4-diazaspiro[4.5]dec-3- en-1-yl}acetamide |
| 16 | 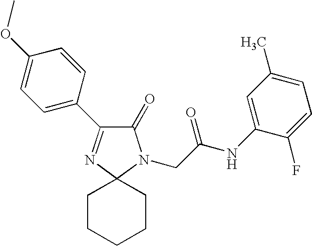 | A | Found 424 (MH⁺) C₂₄H₂₆FN₃O₃ requires 423; 3.45. | N-(2-fluoro-5-methylphenyl)- 2-{3-[4-(methyloxy)phenyl]-2- oxo-1,4-diazaspiro[4.5]dec-3- en-1-yl}acetamide |
| 17 | 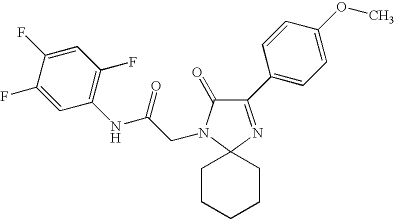 | A | Found 446 (MH⁺) C₂₃H₂₂F₃N₃O₃ requires 445; 3.47. | 2-{3-[4-(methyloxy)phenyl]-2- oxo-1,4-diazaspiro[4.5]dec-3- en-1-yl}-N-(2,4,5- trifluorophenyl)acetamide |
| 18 | 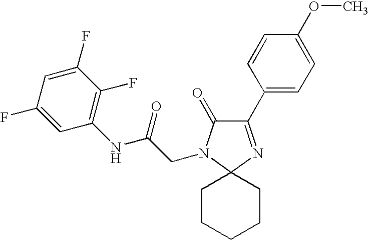 | A | Found 446 (MH⁺) C₂₃H₂₂F₃N₃O₃ requires 445; 3.53. | 2-{3-[4-(methyloxy)phenyl]-2- oxo-1,4-diazaspiro[4.5]dec-3- en-1-yl}-N-(2,3,5- trifluorophenyl)acetamide |
| 19 | 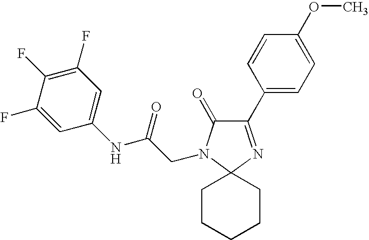 | A | Found 446 (MH⁺) C₂₃H₂₂F₃N₃O₃ requires 445; 3.55. | 2-{3-[4-(methyloxy)phenyl]-2- oxo-1,4-diazaspiro[4.5]dec-3- en-1-yl}-N-(3,4,5- trifluorophenyl)acetamide |
| 20 | 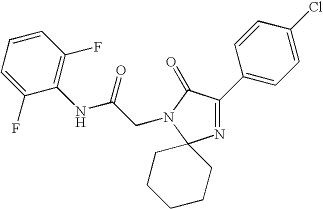 | A | Found 432 (MH⁺) C₂₂H₂₀³⁵ClF₂N₃O₂ requires 431; 3.43. | 2-[3-(4-chlorophenyl)-2-oxo- 1,4-diazaspiro[4.5]dec-3-en- 1-yl]-N-(2,6- difluorophenyl)acetamide |
| 21 | 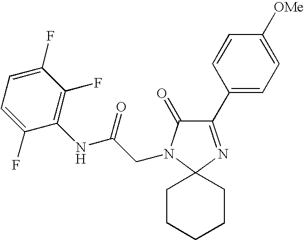 | A | Found 446 (MH+) C23H22F3N3O3 requires 445; 3.26. | 2-{3-[4-(methyloxy)phenyl]-2- oxo-1,4-diazaspiro[4.5]dec-3- en-1-yl}-N-(2,3,6- trifluorophenyl)acetamide |
| 22 | 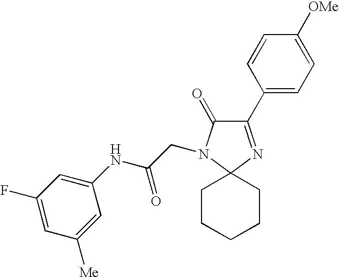 | A | Found 424 (MH⁺) C₂₄H₂₆FN₃O₃ requires 423; 3.47. | N-(3-fluoro-5-methylphenyl)- 2-{3-[4-(methyloxy)phenyl]-2- oxo-1,4-diazaspiro[4.5]dec-3- en-1-yl}acetamide |
| 23 | 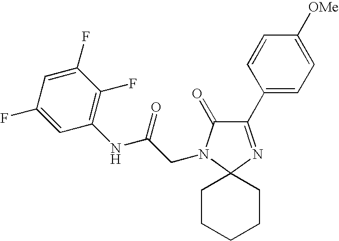 | A | Found 446 (MH⁺) C₂₃H₂₂F₃N₃O₃ requires 445; 3.53. | 2-{3-[4-(methyloxy)phenyl]-2- oxo-1,4-diazaspiro[4.5]dec-3- en-1-yl}-N-(2,3,5- trifluorophenyl)acetamide |
| 24 | 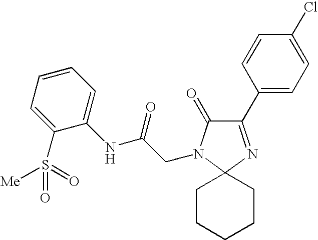 | A | Found 474 (MH⁺) C₂₃H₂₄³⁵ClN₃O₄S requires 473; 3.46. | 2-[3-(4-chlorophenyl)-2-oxo- 1,4-diazaspiro[4.5]dec-3-en- 1-yl]-N-[2- (methylsulfonyl)phenyl] acetamide |
| 25 | 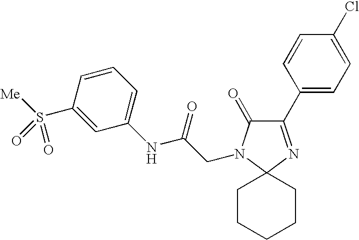 | A | Found 474 (MH⁺) C₂₃H₂₄³⁵ClN₃O₄S requires 473; 3.18. | 2-[3-(4-chlorophenyl)-2-oxo- 1,4-diazaspiro[4.5]dec-3-en- 1-yl]-N-[3- (methylsulfonyl)phenyl] acetamide |
| 26 | 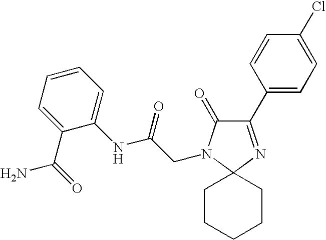 | A | Found 461 (MNa⁺) C₂₃H₂₃³⁵ClN₄O₃ requires 438; 3.17. | 2-({[3-(4-chlorophenyl)-2-oxo- 1,4-diazaspiro[4.5]dec-3-en- 1-yl]acetyl}amino)benzamide |
| 27 | 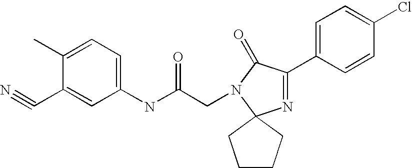 | B | Found 421.2 (MH⁺) C₂₃H₂₁³⁵ClN₄O₂ requires 420; 3.28 min | 2-[3-(4-chlorophenyl)-2-oxo- 1,4-diazaspiro[4.4]non-3-en- 1-yl]-N-(3-cyano-4- methylphenyl)acetamide |
| 28 | 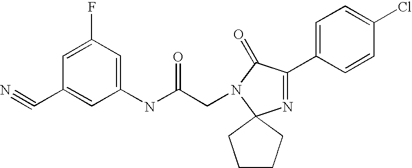 | B | Found 425.2 (MH⁺) C₂₂H₁₈³⁵ClFN₄O₂ requires 424; 3.34 min | 2-[3-(4-chlorophenyl)-2-oxo- 1,4-diazaspiro[4.4]non-3-en- 1-yl]-N-(3-cyano-5- fluorophenyl)acetamide |
| 29 | 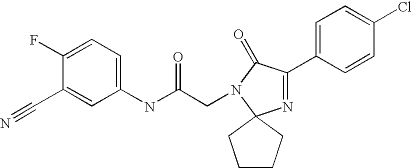 | B | Found 425.2 (MH⁺) C₂₂H₁₈³⁵ClFN₄O₂ requires 424; 3.25 min | 2-[3-(4-chlorophenyl)-2-oxo- 1,4-diazaspiro[4.4]non-3-en- 1-yl]-N-(3-cyano-4- fluorophenyl)acetamide |
| 30 | 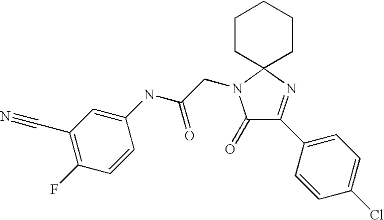 | B | Found 439.0 (MH⁺) C₂₃H₂₀³⁵ClFN₄O₂ requires 438; 1.40 min | 2-[3-(4-chlorophenyl)-2-oxo- 1,4-diazaspiro[4.5]dec-3-en- 1-yl]-N-(3-cyano-4- fluorophenyl)acetamide |
| 31 | 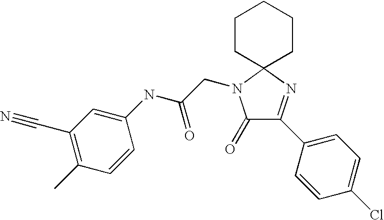 | B | Found 435.0 (MH⁺) C₂₄H₂₃³⁵ClN₄O₂ requires 434; 1.41 min | 2-[3-(4-chlorophenyl)-2-oxo- 1,4-diazaspiro[4.5]dec-3-en- 1-yl]-N-(3-cyano-4- methylphenyl)acetamide |
| 32 | 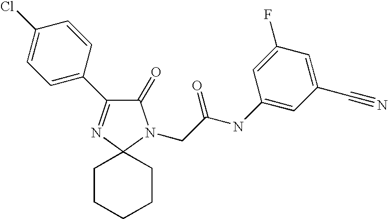 | B | Found 439.0 (MH⁺) C₂₃H₂₀³⁵ClFN₄O₂ requires 438; 1.43 min | 2-[3-(4-chlorophenyl)-2-oxo- 1,4-diazaspiro[4.5]dec-3-en- 1-yl]-N-(3-cyano-5- fluorophenyl)acetamide |

3-Amino-5-(trifluoromethyl)benzonitrile was prepared as described by G. Butora et al. PCT WO2004/041161A2.

1-22. (canceled)

23. A compound of formula (Ia) or a salt or solvate thereof: 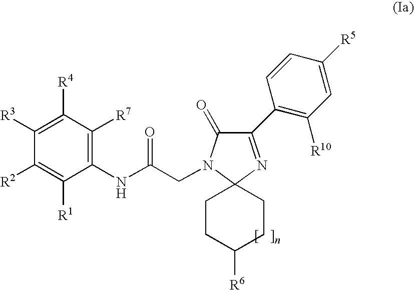
wherein:
R¹ is H, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl or halo;
R² is H, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl or halo;
R³ is H, C₁-C₄alkyl, C₁-C₄alkoxy, haloC₁-C₄alkyl, haloC₁-C₄alkoxy, cyano or halo; or R² and R³ together form a group which is —O—CH₂—O— or —O—CH₂—CH₂—O—;
R⁵ is H, C₁-C₄alkyl, C₁-C₄alkoxy, chloro or fluoro;
R⁶ is H or methyl;
R⁷ is hydrogen, chloro, fluoro, C₁-C₄alkyl, CF₃ or CONR⁸R⁹ wherein R⁸ and R⁹ are independently hydrogen or C₁-C₄alkyl, or R⁸ and R⁹, together with the nitrogen atom to which they are attached, form a 4- to 7-membered ring;
n is 0, 1 or 2;
R¹⁰ is hydrogen or fluoro; and
for R⁴:
(i) when R¹ is chloro, R⁴ is hydrogen, C₁-C₄alkyl, C₁-C₄alkoxy, haloC₁-C₄alkyl, haloC₁-C₄alkoxy, cyano or halo;
(ii) when at least one of R¹, R² and R³ is cyano, haloC₁-C₄alkyl, C₁-C₄alkylsulfonyl or haloC₁-C₄alkoxy, R⁴ is hydrogen, C₁-C₄alkyl, C₁-C₄alkoxy, haloC₁-C₄alkyl, haloC₁-C₄alkoxy, cyano or halo;
(iii) when simultaneously R¹ is hydrogen, R² is hydrogen or methoxy, R³ is hydrogen, methyl, ethyl, methoxy, ethoxy, fluoro or chloro, R⁴ is cyano, haloC₁-C₄alkyl, haloC₁-C₄alkoxy and C₁-C₄alkylsulfonyl;
(iv) when R⁷ is chloro, fluoro, C₁-C₄alkyl, CF₃ or a group CONR⁸R⁹ as defined above, R⁴ is H, C₁-C₄alkyl, C₁-C₄alkoxy, haloC₁-C₄alkyl, haloC₁-C₄alkoxy, cyano or halo;
(v) in all other cases, R⁴ is methyl, chloro, fluoro, cyano, haloC₁-C₄alkyl, or haloC₁-C₄alkoxy;
excluding N-(2-chlorophenyl)-2-[3-(4-fluorophenyl)-2-oxo-1,4-diazaspiro[4.6]undec-3-en-1-yl]acetamide.

24. A compound as claimed in claim 23 wherein R⁶ is hydrogen.

25. A compound as claimed in claim 23 wherein R¹ is hydrogen, methyl, methoxy, trifluoromethyl, trifluoromethoxy, cyano, methylsulfonyl, trifluoromethylsulfonyl or halo.

26. A compound as claimed in claim 23 wherein R² is hydrogen, methyl, methoxy, trifluoromethyl, trifluoromethoxy, cyano, methylsulfonyl, trifluoromethylsulfonyl or halo.

27. A compound as claimed claim 23 wherein R³ is hydrogen, methyl, methoxy, ethoxy, trifluoromethyl, trifluoromethoxy, cyano or halo; or R² and R³ together form a group which is —O—CH₂—O— or —O—CH₂—CH₂—O—.

28. A compound as claimed claim 23 wherein R⁷ is hydrogen, fluoro or CONR⁸R⁹ wherein R⁸ and R⁹ are independently hydrogen or C₁-C₄alkyl.

29. A compound as claimed in claim 23 wherein R¹⁰ is hydrogen.

30. A compound as claimed in claim 23 wherein:
in (i) R⁴ is hydrogen; or
in (ii) R⁴ is hydrogen or haloC₁-C₄alkyl; or
in (iii) R⁴ is haloC₁-C₄alkyl or C₁-C₄alkylsulfonyl; or
in (iv) R⁴ is hydrogen, methyl, methoxy, trifluoromethyl, trifluoromethoxy, cyano or halo; or
in (v) R⁴ is fluoro, cyano, haloC₁-C₄alkyl, or haloC₁-C₄alkoxy.

31. A compound as claimed in claim 23 wherein R⁴ is cyano, haloC₁-C₄alkyl, or haloC₁-C₄alkoxy.

32. A compound as claimed in claim 23 wherein R⁵ is hydrogen, methyl, methoxy, chloro or fluoro.

33. A compound as claimed in claim 32 wherein the compound of formula (Ia) is selected from the group consisting of:
2-[3-(4-chlorophenyl)-2-oxo-1,4-diazaspiro[4.5]dec-3-en-1-yl]-N-(3,5-difluorophenyl)acetamide;
2-{3-[4-(methyloxy)phenyl]-2-oxo-1,4-diazaspiro[4.5]dec-3-en-1-yl}-N-[3-(trifluoromethyl)phenyl]acetamide;
2-[3-(4-chlorophenyl)-2-oxo-1,4-diazaspiro[4.5]dec-3-en-1-yl]-N-(2-cyanophenyl)acetamide;
2-[3-(4-chlorophenyl)-2-oxo-1,4-diazaspiro[4.5]dec-3-en-1-yl]-N-(3-cyanophenyl)acetamide;
2-[3-(4-chlorophenyl)-2-oxo-1,4-diazaspiro[4.5]dec-3-en-1-yl]-N-(4-cyanophenyl)acetamide;
2-[3-(4-chlorophenyl)-2-oxo-1,4-diazaspiro[4.5]dec-3-en-1-yl]-N-[2-chloro-3-(trifluoromethyl)phenyl]acetamide;
2-[3-(4-chlorophenyl)-2-oxo-1,4-diazaspiro[4.5]dec-3-en-1-yl]-N-[3-(trifluoromethyl)phenyl]acetamide;
2-[3-(4-chlorophenyl)-2-oxo-1,4-diazaspiro[4.5]dec-3-en-1-yl]-N-(2,3-dichlorophenyl)acetamide;
N-(3-cyanophenyl)-2-{3-[4-(methyloxy)phenyl]-2-oxo-1,4-diazaspiro[4.5]dec-3-en-1-yl}acetamide;
2-[3-(4-chlorophenyl)-2-oxo-1,4-diazaspiro[4.5]dec-3-en-1-yl]-N-(2,5-difluorophenyl)acetamide;
N-(3,5-difluorophenyl)-2-{3-[4-(methyloxy)phenyl]-2-oxo-1,4-diazaspiro[4.5]dec-3-en-1-yl}acetamide;
N-(3,5-difluorophenyl)-2-{3-[4-(methyloxy)phenyl]-2-oxo-1,4-diazaspiro[4.4]non-3-en-1-yl}acetamide;
N-[3,5-bis(trifluoromethyl)phenyl]-2-{3-[4-(methyloxy)phenyl]-2-oxo-1,4-diazaspiro[4.5]dec-3-en-1-yl}acetamide;
N-[3-cyano-5-(trifluoromethyl)phenyl]-2-{3-[4-(methyloxy)phenyl]-2-oxo-1,4-diazaspiro[4.5]dec-3-en-1-yl}acetamide;
N-[3-bromo-5-(trifluoromethyl)phenyl]-2-{3-[4-(methyloxy)phenyl]-2-oxo-1,4-diazaspiro[4.5]dec-3-en-1-yl}acetamide;
N-(2-fluoro-5-methylphenyl)-2-{3-[4-(methyloxy)phenyl]-2-oxo-1,4-diazaspiro[4.5]dec-3-en-1-yl}acetamide;
2-{3-[4-(methyloxy)phenyl]-2-oxo-1,4-diazaspiro[4.5]dec-3-en-1-yl}-N-(2,4,5-trifluorophenyl)acetamide;
2-{3-[4-(methyloxy)phenyl]-2-oxo-1,4-diazaspiro[4.5]dec-3-en-1-yl}-N-(2,3,5-trifluorophenyl)acetamide;
2-{3-[4-(methyloxy)phenyl]-2-oxo-1,4-diazaspiro[4.5]dec-3-en-1-yl}-N-(3,4,5-trifluorophenyl)acetamide;
N-(2-chlorophenyl)-2-[3-(4-fluorophenyl)-2-oxo-1,4-diazaspiro[4.6]undec-3-en-1-yl]acetamide;
2-[3-(4-chlorophenyl)-2-oxo-1,4-diazaspiro[4. 5]dec-3-en-1-yl]-N-(2,6-difluorophenyl)acetamide;
2-{3-[4-(methyloxy)phenyl]-2-oxo-1,4-diazaspiro[4.5]dec-3-en-1-yl}-N-(2,3,6-trifluorophenyl)acetamide;
N-(3-fluoro-5-methylphenyl)-2-{3-[4-(methyloxy)phenyl]-2-oxo-1,4-diazaspiro[4.5]dec-3-en-1-yl}acetamide;
2-{3-[4-(methyloxy)phenyl]-2-oxo-1,4-diazaspiro[4.5]dec-3-en-1-yl}-N-(2,3,5-trifluorophenyl)acetamide;
2-[3-(4-chlorophenyl)-2-oxo-1,4-diazaspiro[4.5]dec-3-en-1-yl]-N-[2-(methylsulfonyl)phenyl]acetamide;
2-[3-(4-chlorophenyl)-2-oxo-1,4-diazaspiro[4.5]dec-3-en-1-yl]-N-[3-(methylsulfonyl)phenyl]acetamide;
2-({[3-(4-chlorophenyl)-2-oxo-1,4-diazaspiro[4.5]dec-3-en-1-yl]acetyl}amino)benzamide;
2-[3-(4-chlorophenyl)-2-oxo-1,4-diazaspiro[4.4]non-3-en-1-yl]-N-(3-cyano-4-methylphenyl)acetamide;
2-[3-(4-chlorophenyl)-2-oxo-1,4-diazaspiro[4.4]non-3-en-1-yl]-N-(3-cyano-5-fluorophenyl)acetamide;
2-[3-(4-chlorophenyl)-2-oxo-1,4-diazaspiro[4.4]non-3-en-1-yl]-N-(3-cyano-4-fluorophenyl)acetamide;
2-[3-(4-chlorophenyl)-2-oxo-1,4-diazaspiro[4.5]dec-3-en-1-yl]-N-(3-cyano-4-fluorophenyl)acetamide;
2-[3-(4-chlorophenyl)-2-oxo-1,4-diazaspiro[4.5]dec-3-en-1-yl]-N-(3-cyano-4-methylphenyl)acetamide; and
2-[3-(4-chlorophenyl)-2-oxo-1,4-diazaspiro[4.5]dec-3-en-1-yl]-N-(3-cyano-5-fluorophenyl)acetamide;
and salts and solvates thereof.

34. A method of treating a human suffering from or susceptible to schizophrenia, dementia or attention deficit disorder which comprises administering to a patient in need thereof an effective amount of a compound of formula (I): 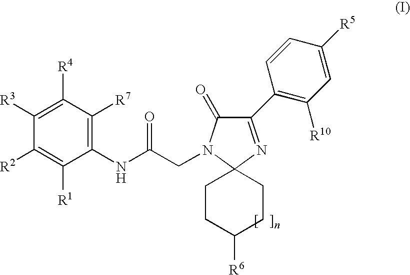
wherein:
R¹ is hydrogen, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl or halo;
R² is hydrogen, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl or halo;
R³ is hydrogen, C₁-C₄alkyl, C₁-C₄alkoxy, haloC₁-C₄alkyl, haloC₁-C₄alkoxy, cyano or halo;
or R² and R³ together form a group selected from —O—CH₂—O— or —O—CH₂—CH₂—O—;
R⁵ is hydrogen, C₁-C₄alkyl, C₁-C₄alkoxy, chloro or fluoro;
R⁶ is hydrogen or methyl;
R⁷ is hydrogen, chloro, fluoro, C₁-C₄alkyl, CF₃ or CONR⁸R⁹ wherein R⁸ and R⁹ are independently hydrogen or C₁-C₄alkyl, or R⁸ and R⁹, together with the nitrogen atom to which they are attached, form a 4- to 7-membered ring;
n is 0, 1 or 2;
R¹⁰ is hydrogen or fluoro; and
R⁴:
(i) when R¹ is chloro or R⁷ is CONR⁸R⁹, then R⁴ is hydrogen, C₁-C₄alkyl, C₁-C₄alkoxy, haloC₁-C₄alkyl, haloC₁-C₄alkoxy, cyano or halo;
(ii) when at least one of R¹, R² and R³ is the group consisting of cyano, haloC₁-C₄alkyl, C₁-C₄alkylsulfonyl or haloC₁-C₄alkoxy, R⁴ is hydrogen, C₁-C₄alkyl, C₁-C₄alkoxy, haloC₁-C₄alkyl, haloC₁-C₄alkoxy, cyano or halo;
(iii) when simultaneously R¹ is hydrogen, R² is hydrogen or methoxy, R³ is hydrogen, methyl, ethyl, methoxy, ethoxy, fluoro or chloro, R⁴ is cyano, haloC₁-C₄alkyl, haloC₁-C₄alkoxy or C₁-C₄alkylsulfonyl;
(iv) when R¹ is C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl or halo, and R⁷ is chloro, fluoro, C₁-C₄alkyl, CF₃ or CONR⁸R⁹, then R⁴ is hydrogen, C₁-C₄alkyl, C₁-C₄alkoxy, haloC₁-C₄alkyl, haloC₁-C₄alkoxy, cyano or halo;
(v) in all other cases, R⁴ is methyl, chloro, fluoro, cyano, haloC₁-C₄alkyl, or haloC₁-C₄alkoxy;
or a pharmaceutically acceptable salt thereof.

35. A pharmaceutical composition comprising a compound of formula (I) or a pharmaceutically acceptable salt thereof and at least one carrier, diluent or excipient wherein formula (I) comprises: 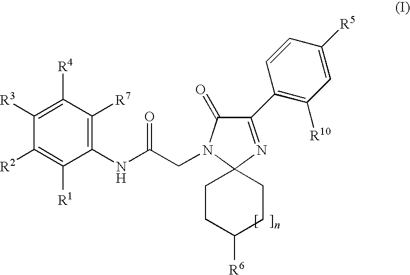
wherein:
R¹ is hydrogen, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl or halo;
R² is hydrogen, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl or halo;
R³ is hydrogen, C₁-C₄alkyl, C₁-C₄alkoxy, haloC₁-C₄alkyl, haloC₁-C₄alkoxy, cyano or halo;
or R² and R³ together form a group selected from —O—CH₂—O— or —O—CH₂—CH₂—O—;
R⁵ is hydrogen, C₁-C₄alkyl, C₁-C₄alkoxy, chloro or fluoro;
R⁶ is hydrogen or methyl;
R⁷ is hydrogen, chloro, fluoro, C₁-C₄alkyl, CF₃ or CONR⁸R⁹ wherein R⁸ and R⁹ are independently hydrogen or C₁-C₄alkyl, or R⁸ and R⁹, together with the nitrogen atom to which they are attached, form a 4- to 7-membered ring;
n is 0, 1 or 2;
R¹⁰ is hydrogen or fluoro; and
R⁴:
(i) when R¹ is chloro or R⁷ is CONR⁸R⁹, then R⁴ is hydrogen, C₁-C₄alkyl, C₁-C₄alkoxy, haloC₁-C₄alkyl, haloC₁-C₄alkoxy, cyano or halo;
(ii) when at least one of R¹, R² and R³ is the group consisting of cyano, haloC₁-C₄alkyl, C₁-C₄alkylsulfonyl or haloC₁-C₄alkoxy, R⁴ is hydrogen, C₁-C₄alkyl, C₁-C₄alkoxy, haloC₁-C₄alkyl, haloC₁-C₄alkoxy, cyano or halo;
(iii) when simultaneously R¹ is hydrogen, R² is hydrogen or methoxy, R³ is hydrogen, methyl, ethyl, methoxy, ethoxy, fluoro or chloro, R⁴ is cyano, haloC₁-C₄alkyl, haloC₁-C₄alkoxy or C₁-C₄alkylsulfonyl;
(iv) when R¹ is C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl or halo, and R⁷ is chloro, fluoro, C₁-C₄alkyl, CF₃ or CONR⁸R⁹, then R⁴ is hydrogen, C₁-C₄alkyl, C₁-C₄alkoxy, haloC₁-C₄alkyl, haloC₁-C₄alkoxy, cyano or halo;
(v) in all other cases, R⁴ is methyl, chloro, fluoro, cyano, haloC₁-C₄alkyl, or haloC₁-C₄alkoxy.

36. A process for the manufacture of a compound of formula (I) (I): 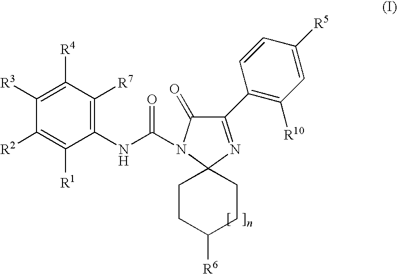
wherein:
R¹ is hydrogen, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl or halo;
R² is hydrogen, C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl or halo;
R³ is hydrogen, C₁-C₄alkyl, C₁-C₄alkoxy, haloC₁-C₄alkyl, haloC₁-C₄alkoxy, cyano or halo;
or R² and R³ together form a group selected from —O—CH₂—O— or —O—CH₂—CH₂—O—;
R⁵ is hydrogen, C₁-C₄alkyl, C₁-C₄alkoxy, chloro or fluoro;
R⁶ is hydrogen or methyl;
R⁷ is hydrogen, chloro, fluoro, C₁-C₄alkyl, CF₃ or CONR⁸R⁹ wherein R⁸ and R⁹ are independently hydrogen or C₁-C₄alkyl, or R⁸ and R⁹, together with the nitrogen atom to which they are attached, form a 4- to 7-membered ring;
n is 0, 1 or 2;
R¹⁰ is hydrogen or fluoro; and
R⁴:
(i) when R¹ is chloro or R⁷ is CONR⁸R⁹, then R⁴ is hydrogen, C₁-C₄alkyl, C₁-C₄alkoxy, haloC₁-C₄alkyl, haloC₁-C₄alkoxy, cyano or halo;
(ii) when at least one of R¹, R² and R³ is the group consisting of cyano, haloC₁-C₄alkyl, C₁-C₄alkylsulfonyl or haloC₁-C₄alkoxy, R⁴ is hydrogen, C₁-C₄alkyl, C₁-C₄alkoxy, haloC₁-C₄alkyl, haloC₁-C₄alkoxy, cyano or halo;
(iii) when simultaneously R¹ is hydrogen, R² is hydrogen or methoxy, R³ is hydrogen, methyl, ethyl, methoxy, ethoxy, fluoro or chloro, R⁴ is cyano, haloC₁-C₄alkyl, haloC₁-C₄alkoxy or C₁-C₄alkylsulfonyl;
(iv) when R¹ is C₁-C₄alkyl, C₁-C₄alkoxy, haloC₁-C₄alkyl, haloC₁-C₄alkoxy, cyano, C₁-C₄alkylsulfonyl, haloC₁-C₄alkylsulfonyl or halo, and R⁷ is chloro, fluoro, C₁-C₄alkyl, CF₃ or CONR⁸R⁹, then R⁴ is hydrogen, C₁-C₄alkyl, C₁-C₄alkoxy, haloC₁-C₄alkyl, haloC₁-C₄alkoxy, cyano or halo;
(v) in all other cases, R⁴ is methyl, chloro, fluoro, cyano, haloC₁-C₄alkyl, or haloC₁-C₄alkoxy; the process comprising:
(a) reacting a compound of formula (II): 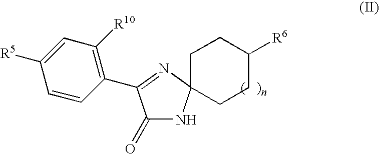
wherein R⁵, R⁶, R¹⁰ and n are as defined for formula (I), with a compound of formula (III): 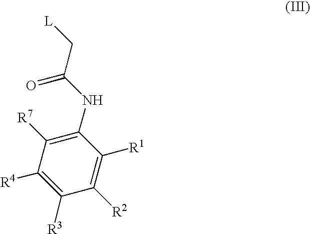
wherein R¹, R², R³, R⁴ and R⁷ are as defined for formula (I) and L is a leaving group; or
(b) reacting a compound of formula (XV): 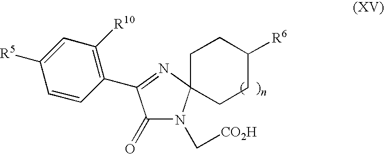
wherein R⁵, R⁶, R¹⁰ and n are as defined for formula (I), with a compound of formula (XVI): 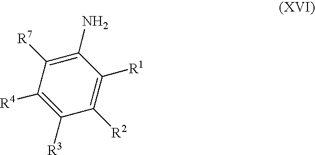
wherein R¹, R², R³, R⁴ and R⁷ are as defined for formula (I); or
(c) reacting a compound of formula (XVII): 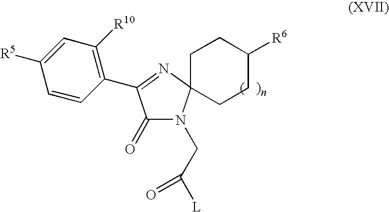
wherein R⁵, R⁶ and R¹⁰ are as defined in formula (I) and L represents a leaving group, with a compound of formula (XVI) as defined in process (b); and thereafter optionally:
removing any protecting groups and/or
forming a salt or solvate.
